SEQUENCE LISTING (8 sequences)

SEQ ID NO: 1 - protein, 868 aa, Homo sapiens
  Cytosolic phospholipase A2 epsilon (Isoform 1)
MSLQASEGCPGLGTNVFVPQSPQTDEEGSRSGRSFSEFEDTQDLDTPGLPPFCPMAPWGSEEGLSPCHLLTVRVIRMKNVRQADMLSQTDCFVSLWLPTASQKKLRTRTISNCPNPEWNESFNFQIQSRVKNVLELSVCDEDTVTPDDHLLTVLYDLTKLCFRKKTHVKFPLNPQGMEELEVEFLLEESPSPPETLVTNGVLVSRQVSCLEVHAQSRRRRKREKMKDLLVMVNESFENTQRVRPCLEPCCPTSACFQTAACFHYPKYFQSQVHVEVPKSHWSCGLCCRSRKKGPISQPLDCLSDGQVMTLPVGESYELHMKSTPCPETLDVRLGFSLCPAELEFLQKRKVVVAKALKQVLQLEEDLQEDEVPLIAIMATGGGTRSMTSMYGHLLGLQKLNLLDCASYITGLSGATWTMATLYRDPDWSSKNLEPAIFEARRHVVKDKLPSLFPDQLRKFQEELRQRSQEGYRVTFTDFWGLLIETCLGDERNECKLSDQRAALSCGQNPLPIYLTINVKDDVSNQDFREWFEFSPYEVGLQKYGAFIPSELFGSEFFMGRLVKRIPESRICYMLGLWSSIFSLNLLDAWNLSHTSEEFFHRWTREKVQDIEDEPILPEIPKCDANILETTVVIPGSWLSNSFREILTHRSFVSEFHNFLSGLQLHTNYLQNGQFSRWKDTVLDGFPNQLTESANHLCLLDTAFFVNSSYPPLLRPERKADLIIHLNYCAGSQTKPLKQTCEYCTVQNIPFPKYELPDENENLKECYLMENPQEPDAPIVTFFPLINDTFRKYKAPGVERSPEELEQGQVDIYGPKTPYATKELTYTEATFDKLVKLSEYNILNNKDTLLQALRLAVEKKKRLKGQCPS

SEQ ID NO: 2 - DNA, 2607 bp, Homo sapiens
  Nucleotide Sequence encoding human cPLA2e isoform 1
atgagtctccaggcctcggaaggctgtcctggcctgggaactaatgtgtttgtcccacagagcccacaaacggatgaagaaggcagcaggtcaggaagaagtttcagtgagttcgaggatacacaggacctggacactcctggtctcccacctttctgtcctatggctccttggggctctgaggaggggctgtctccatgccacctgttgacagtgagggtcatccggatgaaaaatgtccggcaggctgatatgctgagccagacagactgttttgtgagcctctggctgcccaccgcctctcagaagaagctgaggacaaggaccatctccaactgcccaaatccagagtggaatgaaagcttcaacttccagatccagagccgagtgaagaacgtgctagagttgagtgtctgtgatgaagacacagtgacaccagatgaccatctcctgacagttctctatgacctcaccaagctctgtttccgaaagaaaacccacgtgaagtttccactcaacccgcagggcatggaagagctggaggtggagttcctgctggaggagagtccctctccacctgagaccctcgtcaccaatggcgtgctggtgtctcgacaagtctcctgcctggaggttcatgcacaatccaggaggcggaggaagagggagaaaatgaaggacctcctggtgatggtgaacgaatcctttgagaacacccagcgtgtccggccctgcttggaaccctgctgcccaacctctgcctgcttccaaaccgctgcctgcttccactaccccaagtacttccagtcccaggtgcacgtggaagtgcccaagagtcactggagctgtgggctttgctgccgctctcgcaagaagggccccatcagccagcccctcgactgcctttccgatggtcaggtgatgaccctgcctgtgggtgagagttatgaattacacatgaagtctacaccctgccctgagacactggacgtgcggctgggcttcagcctgtgcccagcagagctggagtttctgcagaagcggaaggtcgtggtggccaaggccctgaagcaggtgctgcagctggaggaagacctgcaggaggacgaggtgccgctgatagccatcatggccactgggggtggaacaagatccatgacctccatgtatggccacctgctggggctgcagaagctgaacctcctggactgtgccagctacatcaccggtctatcaggggccacctggaccatggctaccttgtaccgtgaccctgactggtcctccaaaaacttggagcctgctatctttgaggctcggagacatgtggtaaaggacaagctaccctccctgttcccagaccagctccgcaaattccaggaggagctccggcagcgcagccaggaaggctacagggtcacctttacagacttctggggcctgctgatagagacctgcctgggggacgagagaaatgaatgcaaactgtcagatcagcgtgctgctttgagctgcggccagaaccccctgcccatctacctcaccatcaatgtcaaggatgatgtaagcaaccaggacttcagagagtggttcgagttctccccctacgaggtgggcctgcagaagtatggggccttcatcccctccgagctcttcggctccgagttcttcatggggcggctggtgaagaggatcccggagtctcgaatctgctacatgctaggcctgtggagcagcatcttctccctgaacctgctggatgcctggaacctgtcacacacctcggaggagtttttccacaggtggacaagggagaaagtgcaggacatcgaagacgagccgatcctgcctgaaatccccaaatgtgatgctaacatcctggagaccacggtagtgatcccagggtcatggctgtccaattctttccgagaaatccttacccatcggtccttcgtgtctgagtttcacaacttcctgtctgggctgcagctgcacaccaactacctccagaatggccagttctctaggtggaaagacacagtgctagatggtttcccaaaccagctgaccgagtccgcgaaccacctgtgcctgctggacactgcgttctttgtcaactccagctacccgcccctcctcaggccagagcgaaaagccgacctcatcatccacctcaactactgtgctgggtcccagacaaagcccctgaaacaaacctgtgagtactgcactgtgcagaacatccccttccccaaatacgagctgccagatgagaatgaaaatctcaaggaatgctacctgatggagaacccccaggaacccgatgcccccatcgtgactttcttcccactcatcaatgacactttccgaaaatacaaggcaccaggtgtagagcgaagccctgaggagctggagcagggccaggtggacatttatggtcccaaaactccctatgccaccaaggagctgacatacacagaggccacctttgacaagctggtgaaactctcagagtataacatcctgaataataaggacactctcctccaggctctgcggctcgcagtggagaagaagaagcgcctgaagggccagtgtccctcctag

SEQ ID NO: 3 - protein, 492 aa, Homo sapiens
  Isoform 2 of Cytosolic phospholipase A2 epsilon
MATGGGTRSMTSMYGHLLGLQKLNLLDCASYITGLSGATWTMATLYRDPDWSSKNLEPAIFEARRHVVKDKLPSLFPDQLRKFQEELRQRSQEGYRVTFTDFWGLLIETCLGDERNECKLSDQRAALSCGQNPLPIYLTINVKDDVSNQDFREWFEFSPYEVGLQKYGAFIPSELFGSEFFMGRLVKRIPESRICYMLGLWSSIFSLNLLDAWNLSHTSEEFFHRWTREKVQDIEDEPILPEIPKCDANILETTVVIPGSWLSNSFREILTHRSFVSEFHNFLSGLQLHTNYLQNGQFSRWKDTVLDGFPNQLTESANHLCLLDTAFFVNSSYPPLLRPERKADLIIHLNYCAGSQTKPLKQTCEYCTVQNIPFPKYELPDENENLKECYLMENPQEPDAPIVTFFPLINDTFRKYKAPGVERSPEELEQGQVDIYGPKTPYATKELTYTEATFDKLVKLSEYNILNNKDTLLQALRLAVEKKKRLKGQCPS

SEQ ID NO: 4 - DNA, 1479 bp, Homo sapiens
  Nucleotide Sequence encoding human cPLA2e isoform 2
atggccactgggggtggaacaagatccatgacctccatgtatggccacctgctggggctgcagaagctgaacctcctggactgtgccagctacatcaccggtctatcaggggccacctggaccatggctaccttgtaccgtgaccctgactggtcctccaaaaacttggagcctgctatctttgaggctcggagacatgtggtaaaggacaagctaccctccctgttcccagaccagctccgcaaattccaggaggagctccggcagcgcagccaggaaggctacagggtcacctttacagacttctggggcctgctgatagagacctgcctgggggacgagagaaatgaatgcaaactgtcagatcagcgtgctgctttgagctgcggccagaaccccctgcccatctacctcaccatcaatgtcaaggatgatgtaagcaaccaggacttcagagagtggttcgagttctccccctacgaggtgggcctgcagaagtatggggccttcatcccctccgagctcttcggctccgagttcttcatggggcggctggtgaagaggatcccggagtctcgaatctgctacatgctaggcctgtggagcagcatcttctccctgaacctgctggatgcctggaacctgtcacacacctcggaggagtttttccacaggtggacaagggagaaagtgcaggacatcgaagacgagccgatcctgcctgaaatccccaaatgtgatgctaacatcctggagaccacggtagtgatcccagggtcatggctgtccaattctttccgagaaatccttacccatcggtccttcgtgtctgagtttcacaacttcctgtctgggctgcagctgcacaccaactacctccagaatggccagttctctaggtggaaagacacagtgctagatggtttcccaaaccagctgaccgagtccgcgaaccacctgtgcctgctggacactgcgttctttgtcaactccagctacccgcccctcctcaggccagagcgaaaagccgacctcatcatccacctcaactactgtgctgggtcccagacaaagcccctgaaacaaacctgtgagtactgcactgtgcagaacatccccttccccaaatacgagctgccagatgagaatgaaaatctcaaggaatgctacctgatggagaacccccaggaacccgatgcccccatcgtgactttcttcccactcatcaatgacactttccgaaaatacaaggcaccaggtgtagagcgaagccctgaggagctggagcagggccaggtggacatttatggtcccaaaactccctatgccaccaaggagctgacatacacagaggccacctttgacaagctggtgaaactctcagagtataacatcctgaataataaggacactctcctccaggctctgcggctcgcagtggagaagaagaagcgcctgaagggccagtgtccctcctag

SEQ ID NO: 5 - DNA, 2673 bp, Artificial Sequence
  Nucleotide Sequence encoding murine PLA2G4E fused to a flag sequence
atgcagtctattccacactccgatgaagcagacgtggctgggatgacccacgcctcagaaggccaccatggcctggggaccagcatgcttgtcccaaagaacccacaaggggaagaagacagcaagctaggaagaaactgcagtggatttgaagatgcacaggacccacagactgctgtgccctcctcacctttactttccatggcttcttgcagttctcaggaggggtcatctccatgccatctgttgacagtgaggatcattggcatgaaaaacgtccggcaggctgatatactgagtcagacagactgctttgtgaccctctggctgcctactgcctctcagaagaagctgaagaccagaaccatctccaactgcctacacccagagtgggacgaaagcttcacctttcagatccagactcaagtaaagaatgtgctagagctgagcgtctgtgacgaagacaccctgacacaaaatgaccatctcttgacagtcctctatgacctctctaagctttgcctccggaataaaacccatgtgaagttcccactcaacccagagggcatggaagaactggaggtggagttcctactcgaagagaatttctcctcatcagagaccctcatcaccaacggcgtgctggtgtctcgccaagtctcttgcctggaggttcatgcagaatccaggaggccgaggaagaggaagaaaaacaaagaccttctggtgatggtgacagactccttcgagaacacccagcgtgtcccgccttgccaggagccctgctaccccaattctgcctgcttccactaccccaagtactcccagccacagctttacgcagaggcgcctaagagccactgtaactttaggctttgctgctgcggaacacacaggaatgaccctgtctgccagcccctcaattgcctttctgatggccaggtgacaaccctgcctgtgggagagaactatgagctacacatgaagtcctcaccctgctctgacacactggatgtgcggcttggattcagcctgtgccaggaagaggtggagtttgtgcagaagcggaagatggtggtggccaagacactaagtcagatgctgcagctggaggaaggcctgcatgaggatgaggtaccgataatagccatcatggccacaggaggtggcacaaggtctatggtctccttgtatggccacctgctggggttgcagaagctgaactttctggacgcttctacttacatcaccggcttgtcaggtgcaacctggactatggctaccttgtacagtgatcctgagtggtcctccaaaaacctggagactgttgtctttgaggcccggagacatgttgtcaaagacaagatgcctgccctgttcccagatcagctctacaaatggcgagaggacctccaaaagcatagccaggagggctataagaccacgtttacagacttttggggcaagctgatcgagtacagtctgggagataaaaaaaacgaatgcaagctgtcagatcagcgagctgctctgtgcaggggacagaaccctctgcccatctacctcaccatcaatgtcaaggatgatgtaagcaaccaggatttcagagaatggttcgagttctccccctacgaggtgggcatgcagaagtacggagccttcatccccagcgagttatttggctccgagttcttcatggggcggctgatgaagaggattcctgagccggagatgtgctacatgctagggttgtggagtagcatcttttccctgaacctgcttgatgcctggaatttgtctcacacctcagaggagtttttctataggtggacaagggagagactgcatgacatcgaagatgatcccatcctgcctgaaatccctaggtgtgacgataaccccctagagaccacagtagtgatcccaacgacatggctgtccaacaccttccgagaaatcctcacacgcaggcccttcgtgtctgagttccacaacttcctgtacgggatgcagctgcatactgactacttacagaacaggcagttctctatgtggaaagacacagtactggacaccttcccaaaccagctgacacagtttgcaaaacacctgaacctgctggacactgcgttctttgtcaactccagctacgcacccctccttaggccagagagaaaagtcgaccttatcatccacctcaattactgcgcaggatcccagacaaagcccctgaaacaaacctgtgagtactgtaccgagcagaagatccccttccccagcttctccatcctggaagatgacaacagtctcaaggagtgctacgtgatggagaatccccaggagcccgacgcccccatcgtggcttacttcccactcatcagtgacaccttccagaagtacaaggctccaggtgtagagcgaagtcctgacgagctggaactgggccagctgaacatctatggaccaaagtctccctatgccaccaaggagctgacgtacacagaggccgccttcgacaagctggtgaagctctcagaatataatatcctcaataacagagataagctcattcaggccttgagactagcaatggagaagaaacgcatgaggagccagtgtccctccgcggccgcaggaggtggaggtgactacaaggatgacgatgacaagtga

SEQ ID NO: 6 - DNA, 21 bp, Artificial Sequence
  Forward primer fwPLA2G4E
atggtgacagactccttcgag

SEQ ID NO: 7 - DNA, 19 bp, Artificial Sequence
  Reverse primer rvPLA2G4E
cctctgcgtaaagctgtgg

SEQ ID NO: 8 - DNA, 45 bp, Artificial Sequence
  shRNA for PLA2G4E (shPLA2G4E)
ggtctatggtctccttgtatcaagagtacaaggagaccatagacc

(19) United States
(12) Patent Application Publication
(10) Pub. No.: US 2022/0233722 A1
(43) Pub. Date: Jul. 28, 2022

(54) cPLA2e INDUCING AGENTS AND USES THEREOF

(71) Applicants: FUNDACION PARA LA INVESTIGACION MEDICA APLICADA, Pamplona (ES); UNIVERSIDAD DE NAVARRA, Pamplona (ES)

(72) Inventors: Ana María GARCÍA OSTA, Pamplona (ES); María del Mar CUADRADO TEJEDOR, Pamplona (ES); Marta PÉREZ GONZÁLEZ, Pamplona (ES)

(21) Appl. No.: 17/624,178

(22) PCT Filed: Jun. 30, 2020
(86) PCT No.: PCT/EP2020/068414

§ 371 Date: Dec. 30, 2021

(30) Foreign Application Priority Data
Jul. 2, 2019 (EP) 19382563.5

(51) Int. Cl. A61K 48/00; C12N 9/20; A61P 25/28; C12N 15/86

(52) U.S. Cl. CPC A61K 48/0075; C12N 9/20; C12Y 301/01004; C12N 15/86; A61P 25/28

(57) ABSTRACT

The present invention relates to cPLA2e inducing agents and cPLA2e inducing agents for use as a medicament, particularly for use in the treatment of a cognitive disorder and/or disease associated with a cognitive disorder, for example dementia, and more specifically age-related dementia and/or Alzheimer's disease.

### TECHNICAL FIELD

The present invention relates to cPLA2e inducing agents and cPLA2e inducing agents for use as a medicament, more particularly for use in the treatment of a cognitive disorder and/or disease associated with a cognitive disorder, for example dementia, and more specifically age-related dementia and/or Alzheimer's disease.

### BACKGROUND

Mild cognitive impairment is characterized by deficits in memory, language and/or other essential cognitive functions that do not interfere with an individual's daily life. The condition often evolves towards dementia, which is characterized by a global deterioration of cognitive abilities to an extent that does interfere with daily life.

Alzheimer's disease (AD) constitutes nowadays the main form of dementia in the elderly affecting about 50 million people all over the world. The progressive and irreversible cognitive impairment and memory loss that occurs in AD coupled with the presence of Aβ peptide aggregates and neurofibrillary tangles (NFTs) constitutes the main hallmarks of the disease.

Until now, most of the therapies assayed for AD were focused on targeting one of these two histopathological features, especially Aβ levels. However, the high failure rate of AD trials, more than 99% and the high costs associated with this disease makes essential to investigate AD with the aim of finding new therapies.

Studying AD is becoming complex as sometimes there is discordance between the appearance of the classic AD markers and the symptoms of dementia. Substantial AD lesions have been observed in the brain of cognitively normal elderly subjects in several longitudinal studies. These findings suggest that the classical AD hallmarks are not enough to produce dementia and open the possibility of studying this AD resilient patients in order to find new possible targets for AD treatments.

Cognitive impairment is a condition associated with a large number of brain disorders. Brain disorders can have many causes, e.g., degenerative conditions, heredity, trauma, infection, malnutrition and others. For example, cognitive impairment can be associated with aging and/or neurodegenerative diseases such as Alzheimer's disease, Parkinson's disease, Huntington's disease, Amyotrophic Lateral Sclerosis (ALS), psychosis, Parkinson's disease psychosis, Alzheimer's disease psychosis, Lewy-body dementia, prionic neurodegenerative disorders such as Creutzfeld-Jacob disease and kuru disease, corticobasal degeneration, frontotemporal lobar degeneration, multiple sclerosis, normal pressure hydrocephalus, organic chronic brain syndrome, Pick's disease, progressive supranuclear palsy, or senile dementia. Cognitive impairment may also have a congenital basis, e.g., Prader-Willi syndrome, Down Syndrome, Fragile X Syndrome, Angelman syndrome and autism spectrum disorder. Cognitive impairment may also be associated with trauma to the brain, such as that caused by chronic subdural hematoma, concussion, stroke, intracerebral hemorrhage, or with other injury to the brain, such as that caused by infection (e.g., encephalitis, meningitis, and septicemia) or drug intoxication or abuse. Cognitive impairment may also be associated with other conditions which impair or otherwise affect normal functioning of the central nervous system, including sleep deprivation, psychiatric disorders such as anxiety disorders, dissociative disorders, mood disorders, schizophrenia, treatment with psychiatric medications, treatment with dopamine agonists and somatoform and factitious disorders; it may also be associated with conditions of the peripheral nervous system, such as chronic pain. In some cases, the cause of a cognitive impairment may be unknown or uncertain.

Cognitive impairment can be manifest in many ways, e.g., deficits in learning and/or memory including, but not limited to, attention, information acquisition, information processing, working memory, short-term memory, long-term memory, anterograde memory, retrograde memory, memory retrieval, discrimination learning, decision-making, language retrieval, inhibitory response control, attentional set-shifting, delayed reinforcement learning, reversal learning, the temporal integration of voluntary behavior, and expressing an interest in one's surroundings and self-care. Cognitive impairment may be characterized by progressive loss of memory, cognition, reasoning, executive functioning, planning, judgment and emotional stability

Although many advances have been made, treatments for cognitive impairment associated with brain disorders remain largely inadequate. For diseases such as Huntington's disease, Parkinson's disease, Amyotrophic Lateral Sclerosis, Alzheimer's disease, Prader-Willi syndrome, Lewy body dementia and others, treatments may be limited or unavailable. There is a need for additional therapeutic options for treating cognitive impairment associated with brain disorders.

### SUMMARY

Now Inventors surprisingly disclose that overexpression of hippocampal PLA2G4E (also named cytosolic phospholipase A2 epsilon (cPLA2e) mediated by treatment with AAV2/9-mPLA2G4E (a viral vector encoding cPLA2e), significantly rescued spatial memory impairment in elderly APP/PS1 mice two months after treatment by stereotactic injection; and that it also improved memory retention in elderly C57BL/6/SJL WT mice three months after the stereotactic injection treatment.

Accordingly, in a first aspect the invention relates to a nucleic acid construct that comprises a nucleotide sequence encoding a cytosolic phospholipase A2 epsilon (cPLA2e).

In a particular embodiment of said nucleic acid construct, the cPLA2e is a human cPLA2e; typically human cPLA2e of SEQ ID NO: 1 or SEQ ID NO:3, or a variant human cPLA2e having at least 70%, sequence identity with respect to human cPLA2e SEQ ID NO:1 or SEQ ID NO:3.

In a more particular embodiment of said nucleic acid construct, the nucleotide sequence encoding cPLA2e is SEQ ID NO:2 or SEQ ID NO:4.

In specific embodiments, said nucleic acid construct further comprises a promoter operably-linked to the nucleotide sequence encoding a cPLA2e.

In more specific embodiments of said nucleic acid construct the promoter operably-linked to the nucleotide sequence encoding a cPLA2e is a neuronal-specific promoter; notably said promoter is a SYN1 promoter or hybrid SYN1 promoter.

In specific embodiments, said nucleic acid construct further comprises a polyadenylation signal sequence; notably a polyadenylation signal sequence of bovine growth hormone gene.

In specific embodiments, said nucleic acid construct comprises a 5′ITR and a 3′ITR sequences; preferably a 5′ITR and a 3′ITR sequences of an adeno-associated virus, more preferably a 5′ITR and a 3′ITR sequences from the AAV2 serotype.

In other embodiments, the nucleic acid construct of the invention is an RNA, notably an mRNA.

In an aspect the invention relates to a vector that comprises a nucleic acid construct of the invention; preferably said vector is a viral vector; more preferably an AAV vector.

In an aspect the invention relates to a viral particle that includes a nucleic acid construct of the invention.

In specific embodiments, said viral particle is selected among AAV particles, preferably including capsid proteins selected from the group consisting of AAV2, AAV5, AAV9, and AAV TT serotypes.

In an aspect, the invention also relates to a host cell comprising a nucleic acid construct or an expression vector of the invention.

In a further aspect, the invention relates to a process for producing viral particles comprising:
a) culturing a packaging cell comprising a nucleic acid construct or a vector of the invention in a culture medium; and
b) harvesting the viral particles from the cell culture supernatant and/or inside the cells.

In another aspect, the invention relates to a pharmaceutical composition comprising a nucleic acid construct, a vector, or a viral particle, or a host cell of the invention; and a pharmaceutically acceptable carrier or excipient.

In another aspect, the invention relates to a nucleic acid construct, a vector, a viral particle, or host cell of the invention, or to a pharmaceutical composition comprising said nucleic acid construct, vector, viral particle or host cell for use as a medicament.

In yet another aspect, the invention relates to a cPLA2e inducing agent for use as a medicament.

In a related aspect, the invention relates to a cPLA2e inducing agent for use in the treatment of cognitive disorders and/or diseases associated with cognitive disorders in a subject in need thereof.

In specific embodiments, said disease associated with cognitive disorders is dementia.

In specific embodiments, said disease associated with cognitive disorders is an age-related dementia or an Alzheimer's disease.

In specific embodiments, said cPLA2e inducing agent is selected from the group consisting of a) a nucleic acid construct of the invention; b) a vector comprising a nucleic acid construct of the invention; c) a viral particle comprising a nucleic acid construct or vector of the invention; d) a host cell comprising a nucleic acid construct or vector of the invention; e) a cPLA2e polypeptide or protein; and f) a pharmaceutical composition comprising any of said nucleic acid construct, vector comprising the nucleic acid construct, viral particle comprising the nucleic acid construct or vector, and cPLA2e polypeptide or protein.

In more specific embodiments, said cPLA2e inducing agent is a nucleic acid construct, a vector or a viral particle of the invention, or a pharmaceutical composition comprising said nucleic acid, vector or viral particle.

In specific embodiments, said cPLA2e inducing agent is a protein with cPLA2e activity; preferably a protein of SEQ ID NO:1 or SEQ ID NO:3.

### LEGENDS OF THE FIGURES

FIG. 1A. Escape latency to the hidden-platform in the MWM test for elderly WT (negative control), APP/PS1 sham (sham-injected APP/PS1 mice) and APP/PS1 AAV2/9-mPLA2G4E (APP/PS1 mice treated with AAV2/9-mPLA2G4E) two months after stereotactic surgery. (Two way ANOVA test followed by Bonferroni's post hoc test, n=6-9, *P≤0.05 APP/PS1 sham vs WT, **P≤0.01 APP/PS1 sham vs WT, +P≤0.05 APP/PS1 AAV2/9-mPLA2G4E vs WT, $P≤0.05 APP/PS1 AAV2/9-mPLA2G4E vs APP/PS1 sham and $$P≤0.01 APP/PS1 AAV2/9-mPLA2G4E vs APP/PS1 sham).

FIG. 1B. Percentage of time spent in the correct quadrant during the 15 s and 60 s probe trials on day 6th for elderly WT, APP/PS1 sham and APP/PS1 AAV2/9-mPLA2G4E two months after hippocampal injections. (One way ANOVA test followed by Newman-Kewls post hoc test, n=6-9, *P≤0.05 APP/PS1 sham vs WT, **P≤0.01 APP/PS1 sham vs WT and $P≤0.05 APP/PS1 AAV2/9-mPLA2G4E vs APP/PS1 sham).

FIG. 2A. Representative Golgi staining images of apical dendrites on CA1 hippocampal pyramidal neurons from elderly WT (negative control), APP/PS1 sham (sham-injected APP/PS1 mice) and APP/PS1 AAV2/9-mPLA2G4E (APP/PS1 mice treated with AAV2/9-mPLA2G4E). Scale bar=10 μm.

FIG. 2B. Histoblot showing spine density quantification of CA1 hippocampal pyramidal neurons from WT, APP/PS1 sham and AAV2/9-PLA2G4E treated mice (One way ANOVA test followed by Newman-Kewls post hoc test, n=4, +p≤0.05 WT vs APP/PS1 AAV2/9-PLA2G4E, $p≤0.05 APP/PS1 sham versus APP/PS1 AAV2/9-PLA2G4E).

FIG. 3A. Escape latencies of the hidden-platform in the MWM test for elderly WT sham (sham-injected C57BL/6/SJL WT mice) and WT AAV2/9-mPLA2G4E (C57BL/6/SJL WT mice treated with AAV2/9-mPLA2G4E) 3 months after hippocampal injections by stereotactic surgery (Two way ANOVA test followed by Bonferroni's post hoc test, n=4-5).

FIG. 3B. Percentage of time spent in the correct quadrant during the 15 s and 60 s probe trials on day 5th for elderly WT sham and WT AAV2/9-mPLA2G4E mice 3 months after stereotactic surgery. (One way ANOVA test followed by Newman-Kewls post hoc test, n=4-5, *P≤0.05 WT AAV2/9-mPLA2G4E vs WT sham).

FIG. 4A. Diagram showing the experimental design of the Fear Conditioning paradigm used to elucidate the role of PLA2G4E in memory function. Graph indicating the percentage of freezing behavior of TT mice during the training and test phase respectively

FIG. 4B. pCREB levels measured by immunoblotting in hippocampal extracts and normalized vs β-actin (one-way ANOVA test followed by Newman-Kewls post hoc test, n=7-8, **P≤0.01 Naïve vs TT, ++P≤0.01 T24 vs TT).

FIG. 4C. PLA2G4E levels measured by immunoblotting in hippocampal extracts and normalized vs β-actin (one-way ANOVA test followed by Newman-Kewls post hoc test, n=7-8, **P≤0.01 Naïve vs TT, ++P≤0.01 T24 vs TT).

FIG. 5. Levels of pCREB, pGluA1, synapsin I and PLA2G4E levels were measured by immunoblotting and normalized vs β-actin in primary neuronal cultures after treatment with bicuculline (Bic) and/or AAV9-shPLA2G4E (shPLA) (One way ANOVA test followed by Newman-Kewls post hoc test, n=3-6, **P≤0.01, ***P≤0.001 control versus Bic; ++P≤0.01 control vs shPLA; $P≤0.05, $$P≤0.01, $$$P≤0.001 Bic versus shPLA+Bic). Data are expressed as arbitrary units (mean±SEM) with respect to controls.

### DETAILED DESCRIPTION

In an aspect the invention relates to a cytosolic phospholipase A2 epsilon inducing agent for use as a medicament, and more specifically for use in the treatment of a cognitive disorder and/or a disease associated with a cognitive disorder in a subject in need thereof.

As used herein, the terms “cytosolic phospholipase A2 epsilon”, and “phospholipase A2 group IVE”, “PLA2G4E”, or “cPLA2e” refer interchangeably to a Calcium-dependent enzyme member of the cytosolic phospholipase A2 group IV family that selectively hydrolyzes glycerophospholipids in the sn-2 position. Members of this family are involved in regulation of membrane tubule-mediated transport. This enzyme plays a role in trafficking through the clathrin-independent endocytic pathway. The enzyme regulates the recycling process via formation of tubules that transport internalized clathrin-independent cargo proteins back to the cell surface (Capestrano M. et al. Journal of Cell Science 2014; 127:977-993). PLA2G4E can catalyze the calcium-dependent formation of N-acyl phosphatidylethanolamines (NAPEs) using phosphatidylethanolamine (PE) as the acyl chain donor (Ogura Y. et al. Nat Chem Biol. 2016; 12(9): 669-671). Human cPLA2e is naturally encoded by PLA2G4E gene; human cPLA2e is recorded for example at UniprotKB (https://www.uniprot.org/) with entry Accession number Q3MJ16. This entry describes 2 isoforms produced by alternative splicing: a) isoform 1 (with identifier: Q3MJ16-3) that is chosen as the ‘canonical’ sequence (SEQ ID NO:1); and b) isoform 2 (with identifier: Q3MJ16-2), that differs from the canonical sequence in that amino acids 1-376 are missing in isoform 2 (SEQ ID NO:3). The term “cPLA2e” refers to the enzyme and any additional co-translation or post-translational modifications thereof.

As used herein, the term “cPLA2e inducing agent” refers to an agent (molecule or composition) that when administered to a cell, directly or indirectly produces a gaining of cPLA2e activity within the cell; particularly an agent that produces a gaining in expression of the enzyme cPLA2e, such as a cPLA2e transgene (i.e. a nucleotide sequence encoding a cPLA2e), or an expression product of said transgene.

### The Nucleic Acid Construct

In an embodiment the cPLA2 inducing agent for the use of the invention is or comprises a nucleic acid construct that comprises a nucleotide sequence encoding a cytosolic phospholipase A2 epsilon (cPLA2e).

Thus, in another aspect the invention relates to a nucleic acid construct that comprises a nucleotide sequence encoding a cytosolic phospholipase A2 epsilon (cPLA2e).

The terms “nucleic acid” and “polynucleotide” or “nucleotide sequence” are interchangeably used herein to refer to any molecule composed of or comprising monomeric nucleotides. A nucleic acid may be an oligonucleotide or a polynucleotide. A nucleotide sequence may be a DNA or RNA. A nucleotide sequence may be chemically modified or artificial. Nucleotide sequences include peptide nucleic acids (PNA), morpholinos and locked nucleic acids (LNA), as well as glycol nucleic acids (GNA) and threose nucleic acid (TNA). Each of these sequences is distinguished from naturally-occurring DNA or RNA by changes to the backbone of the molecule. Also, phosphorothioate nucleotides may be used. Other deoxynucleotide analogs include methylphosphonates, phosphoramidates, phosphorodithioates, N3′P5′-phosphoramidates and oligoribonucleotide phosphorothioates and their 2-O-allyl analogs and 2′-O-methylribonucleotide methylphosphonates which may be used in a nucleotide of the invention.

As used herein the term “nucleic acid construct” refers to a non-naturally occurring nucleic acid resulting from the use of recombinant DNA technology. Especially, a nucleic acid construct is a nucleic acid molecule, either single- or double-stranded, which has been modified to contain segments of nucleic acid sequences, which are combined or juxtaposed in a manner which would not otherwise exist in nature.

In some embodiments, the nucleic acid construct of the invention comprises a nucleotide sequence encoding a naturally-occurring cPLA2e (wild type cPLA2e); e.g., a naturally-occurring human cPLA2e (e.g. isoform 1, or 2), a primate, murine or other mammalian known cPLA2e. In some embodiments, the cPLA2e is a variant, a peptide or a polypeptide containing a substitution, and insertion and/or an addition, a deletion and/or a covalent modification with respect to a naturally-occurring cPLA2e, typically with respect to human cPLA2e isoform 1, or 2. In some embodiments, cPLA2e encoded by the nucleic acid construct of the invention is a fusion protein or polypeptide in which some amino acids (e.g. tags) or polypeptides (e.g. a carrier polypeptide), can be added to the encoded cPLA2e (e.g., at the N-terminal or C-terminal ends), e.g., for localization or targeting. In some embodiments, cPLA2e encoded by the nucleic acid construct is a fragment cPLA2e to which amino acid residues located at the carboxy, amino terminal, or internal regions of the cPLA2e, typically human cPLA2e isoform 1, or 2, can optionally be deleted.

In an embodiment, the nucleic acid construct of the present invention comprises a nucleotide sequence encoding a human cPLA2e, preferably a human cPLA2e of SEQ ID NO:1 or SEQ ID NO:3, that correspond respectively to isoform 1 or 2; or a variant human cPLA2e having at least 70%, 75%, 80%, 85%, 90%; 95% or 99% sequence identity with respect to the coding sequence of a naturally-occurring or recombinant cPLA2e; typically with respect to human cPLA2e SEQ ID NO:1 or SEQ ID NO:3.

As recognized by those skilled in the art, human cPLA2e isoform 1, or 2 protein fragments, functional protein domains, variants, and homologous proteins (orthologs) are also considered to be within the scope of the cPLA2e of the nucleic acid construct of the invention. It is understood the different embodiments of the cPLA2e have substantially the same cPLA2e activity as human cPLA2e isoform 1 or 2. Substantially the same activity can be for instance an activity of up to ±5%, including ±4, ±3, ±2%, ±1% or less.

As mentioned-above, cPLA2e is a calcium-dependent enzyme member of the cytosolic phospholipase A2 group IV family that selectively hydrolyzes glycerophospholipids in the sn-2 position. It has been described to exhibit very low phospholipase (PLA) activity. Instead it has been shown to have a calcium-dependent N-acyltransferase (Ca-NAT) activity that produces N-acyl phosphatidylethanolamines (NAPEs) and N-acyl ethanolamines (NAEs) in mammalian cells. Its transacylase properties have been associated to a serine hydrolase activity (Ogura et al., Nat Chem Biol. 2016, 12(9), 669-671).

Ca-NAT activity can be determined by measuring in a biological sample (e.g. cell lysate) the production of NAPEs. For instance, N-C16:0 DOPE production in reactions with DPPC (40 μM) and DOPE (75 μM) for 30 min at 37° C. with or without CaCl₂ (3 mM) added to the reaction mixture. Ca-independent activities are substracted in the calculations of Ca-dependent activity. Alternatively, targeted analysis of NAPEs production (e.g. ¹³C16:0-containing NAPEs) can be conducted by incubating mammalian cells (e.g. HEK293T cells) in a serum-containing culture medium with or without 2 μm ionomycin. Cells are incubated at 37° C. for a period of time (e.g. 30 min) before lipid extraction. Extracted lipids can be chromatographically separated and analysed by mass spectrometry (e.g. LC-MS/MS).

In a preferred embodiment of the nucleic acid construct of the invention, the nucleotide sequence encoding a cPLA2e is SEQ ID NO:2 or SEQ ID NO:4; or a variant nucleotide sequence having at least 75%, at least 80%, at least 85%, at least 90% or at least 95% identity with respect to SEQ ID NO:2 or 4.

As used herein, the term “sequence identity” or “identity” refers to the number of matches (identical nucleic acid residues or amino acid residues) in positions from an alignment of two polynucleotide sequences or two polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithm (e.g. Needleman and Wunsch algorithm; Needleman and Wunsch, 1970, J Mol Biol.; 48(3):443-53) which aligns the sequences optimally over the entire length, while sequences of substantially different lengths are preferably aligned using a local alignment algorithm (e.g. Smith and Waterman algorithm (Smith and Waterman, 1981, J Theor Biol.; 91(2):379-80) or Altschul algorithm (Altschul S F et al., 1997, Nucleic Acids Res.; 25(17):3389-402; Altschul S F et al., 2005, Bioinformatics; 21(8):1451-6). Alignment for purposes of determining percent nucleic acid sequence identity can be achieved in various ways that are within the skill in the art, for instance, using publicly available computer software available on internet web sites such as http://blast.ncbi.nlm.nih.gov/ or http://www.ebi.ac.uk/Tools/emboss/. Those skilled in the art can determine appropriate parameters for measuring alignment, including any algorithms needed to achieve maximal alignment over the full length of the sequences being compared. For purposes herein, % nucleic acid sequence identity values refers to values generated using the pairwise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

The nucleic acid construct described herein may have different uses: among others, it may be used to generate a viral vector for gene therapy; or to generate a non-viral vector also for gene therapy, such as a nucleic acid construct with mRNA structure.

In an embodiment the nucleic acid construct according to the present invention includes a nucleotide sequence encoding a cPLA2e and at least suitable nucleic acid elements for its expression in a host cell.

For example, in an embodiment the nucleic acid construct comprises a nucleotide sequence encoding a cPLA2e and one or more control sequence(s) required for expression of said coding sequence in the relevant target cell types or tissues. Generally, the nucleic acid construct comprises a coding sequence and regulatory sequences preceding (5′ non-coding sequences) and following (3′ non-coding sequences) the coding sequence that are required for expression of the selected gene product. Thus, in specific embodiments, said nucleic acid construct comprises at least (i) a nucleotide sequence encoding a cPLA2e under the control of (ii) a promoter and (iii) a 3′ untranslated region that usually contains a polyadenylation signal sequence and/or transcription terminator. The nucleic acid construct may also comprise additional regulatory elements such as, for example, enhancer sequences, introns, microRNA targeted sequence, a polylinker sequence facilitating the insertion of a DNA fragment within a vector and/or splicing signal sequences.

### The Promoter

In one embodiment, the nucleic acid construct of the invention also comprises a promoter. Said promoter initiates transgene expression upon introduction into a host cell.

As used herein, the term “transgene” refers to nucleic acid molecule, DNA or cDNA encoding a gene product for use as the active principle in gene therapy. The gene product may be an RNA, peptide or protein; transgene may encode a natural gene product or a recombinant non naturally occurring gene product e.g. the cPLA2e.

As used herein, the term “promoter” refers to a regulatory element that directs the transcription of a nucleic acid (transgene) to which it is operably linked. A promoter can regulate both rate and efficiency of transcription of an operably linked nucleic acid. A promoter may also be operably linked to other regulatory elements which enhance (“enhancers”) or repress (“repressors”) promoter-dependent transcription of a nucleic acid. These regulatory elements include, without limitation, transcription factor binding sites, repressor and activator protein binding sites, and any other sequences of nucleotides known to one of skill in the art to act directly or indirectly to regulate the amount of transcription from the promoter, including e.g. attenuators, enhancers, and silencers. The promoter is located near the transcription start site of the gene or coding sequence to which is operably linked, on the same strand and upstream of the DNA sequence (towards the 5′ region of the sense strand). A promoter can be about 100-1000 base pairs long. Positions in a promoter are designated relative to the transcriptional start site for a particular gene (i.e., positions upstream are negative numbers counting back from −1, for example −100 is a position 100 base pairs upstream).

As used herein, the term “operably linked” refers to a linkage of polynucleotide (or polypeptide) elements in a functional relationship. A nucleic acid is “operably linked” when it is placed into a functional relationship with another nucleic acid sequence. For instance, a promoter or transcription regulatory sequence is operably linked to a coding sequence if it affects the transcription of the coding sequence. Operably linked means that the polynucleotide sequences being linked are typically contiguous; where it is necessary to join two protein encoding regions, they are contiguous and in reading frame.

In an embodiment, the nucleic acid construct of the invention further comprises a promoter operably-linked to the nucleotide sequence encoding a cPLA2e.

In an embodiment said promoter operably linked to the cPLA2e coding sequence is an heterologous promoter. As used herein, the term “heterologous” when used as an attribute of a nucleotide or a peptide sequence (e.g. heterologous promoter, heterologous enhancer, and the like) means a sequence that is not naturally operably linked to another nucleotide or peptide sequence; in this particular case, “heterologous promoter” means a promoter sequence that it is not naturally operably linked to a nucleotide sequence encoding a cPLA2e.

Typically, such promoter may be tissue or cell type specific promoter, or an organ-specific promoter, or a promoter specific to multiple organs or a systemic or ubiquitous promoter.

In a particular embodiment, the nucleic acid construct of the invention further comprises a promoter operably-linked to the nucleotide sequence encoding a cPLA2e and wherein said promoter directs the expression of encoded cPLA2e at least in neurons of the hippocampus.

In a particular embodiment of the nucleic acid construct of the invention the promoter operably linked to the nucleotide sequence encoding a cPLA2e is a neuronal-specific promoter.

As used herein, the term “specific promoter” relates to a promoter that is not necessarily restricted in activity to a single cell type but which nevertheless shows selectivity in that it is active in one group of cells or tissues and less active or silent in another group. However, it may be preferred that the promoter of the nucleic acid construct of the invention shows strict cell-specificity in that they are only active at detectable levels in neuronal cells.

Thus, as used herein, a “neuronal-specific promoter” is a promoter that controls expression of genes that are uniquely or predominantly expressed in neuronal cells or in cells derived from neuronal cells. A neuronal-specific promoter directs expression of a gene in neuronal cells or in cells derived from neuronal cells, but does not substantially direct expression of that same gene in other cell types, for example glial cells, thus having neuronal specific transcriptional activity. In some instances there may be some low level expression in other cell types, but such expression is substantially lower than in neuronal cells, for example expression in neuronal cells may be at least 2, at least 3, at least 4, at least 5 or at least 10 times higher than expression levels in other cells. Such a promoter may be a strong promoter or it may be a weak promoter, and it may direct constitutive expression of a gene in a neuronal cell or a cell derived from a neuronal cell, or it may direct expression in response to certain conditions, signals or cellular events.

Accordingly, a neuronal-specific promoter allows an active expression in the neurons of the gene linked to it and prevents its expression in other cells or tissues.

In a more particular embodiment of the nucleic acid construct of the invention the promoter operably linked to the nucleotide sequence encoding a cPLA2e is a neuronal-specific promoter selected from the group consisting of Synapsin 1 (SYN1) gene promoter (Kügler S et al. Gene Ther. 2003; 10(4): 337-47), neuron specific enolase (NSE) gene promoter (Forss-Petters S et al. Neuron. 1990; 5(2):187-97; Twyman R M et a. J Mol Neurosci. 1997; 8(1):63-73), Hb9 gene promoter (Eur J Neurosci. 1997; 9:452; J Neurosci Res. 2000; 59:321), prion protein (Prnp) gene promoter (Weber P et al. Eur J Neurosci. 2001; 14:1777), α-calcium-calmodulin dependent kinase II (CaMKIIα) gene promoter (Dittgen T et al. Proc Natl Acad Sci USA. 2004; 101: 18206-18211), methyl CpG binding protein 2 (MECP2) gene promoter (Adachi M. et al. Hum Mol Genet. 2005; 14(23):3709-3722; Gray S. J. et al. Hum Gene Ther. 2011; 22(9): 1143-1153), and tubulin al (Tal) gene promoter (Gloster A. et al. J Neurosci. 1994; 14:7319).

Typically, such promoter (particularly the neuronal-specific promoter of the selection group described above) may be the complete promoter, which includes core, proximal and distal promoter elements; a fragment of the promoter, e.g. core promoter, or any other fragment sufficient to direct gene expression in target cell, tissue or organ; or a chimeric or hybrid promoter, e.g. a promoter that includes the core promoter of a gene, and heterologous enhancer sequences, from another gene or synthetic. The term “core promoter” refers herein to the minimal portion of the promoter required to properly initiate transcription. It consists of a transcription start site and functional sequences for binding the transcription start complex (TATA-box) inside a cell or a host organism. Non-limiting examples of suitable neuronal-specific hybrid promoters are hybrid promoters based on SYN1 promoter, e.g. hybrid promoters resulting from the fusion of promoter elements of SYN1 and CMV genes (e.g., Matsuzaki Y. et al. J Neurosci Methods 2014; 223:133-143); Hb9 promoter, e.g. mouse Hb9 enhancer fused to Hsp68 minimal promoter (Singh N R et al. Exp Neurol. 2005; 196(2):224-234) or to CMV minimal promoter (Lukashchuk V. et al. Mol Ther Methods Clin Dev. 2016; 3: 15055), among others.

In an embodiment of the nucleic acid construct of the invention the promoter operably linked to the nucleotide sequence encoding a cPLA2e is a SYN1 promoter, or a hybrid SYN1 promoter, such as a hybrid SYN1 promoter that comprises core SYN1 promoter fused to CMV gene promoter elements.

In an embodiment, the nucleic acid construct of the invention, comprises a hybrid SYN1 promoter operably linked to a nucleotide sequence encoding a cPLA2e, typically a cPLA2e of SEQ ID NO:1 or 3; wherein preferably coding nucleotide sequence is SEQ ID NO:2 or 4.

All these promoter sequences have properties of allowing expression of cPLA2e encoded by the nucleic acid construct in at least the neurons of the hippocampus.

In specific embodiments, the promoter for use in the nucleic acid construct of the invention may be a chemical inducible promoter. As used herein, a chemical inducible promoter is a promoter that is regulated by the in vivo administration of a chemical inducer to said subject in need thereof. Examples of suitable chemical inducible promoters include without limitation Tetracycline/Minocycline inducible promoter (Chtarto 2003, Neurosci Lett. 352:155-158) or rapamycin inducible systems (Sanftner 2006, Mol Ther. 13:167-174).

### The Polyadenylation Signal

Nucleic acid construct embodiments may also include a polyadenylation signal sequence; together or not with other optional nucleotide elements. As used herein, the term “polyadenylation signal” or “poly(A) signal” refers to a specific recognition sequence within 3′ untranslated region (3′ UTR) of the gene, which is transcribed into precursor mRNA molecule and guides the termination of the gene transcription. Poly(A) signal acts as a signal for the endonucleolytic cleavage of the newly formed precursor mRNA at its 3′-end, and for the addition to this 3′-end of a RNA stretch consisting only of adenine bases (polyadenylation process; poly(A) tail). Poly(A) tail is important for the nuclear export, translation, and stability of mRNA. In the context of the invention, the polyadenylation signal is a recognition sequence that can direct polyadenylation of mammalian genes and/or viral genes, in mammalian cells.

Poly(A) signals typically consist of a) a consensus sequence AAUAAA, which has been shown to be required for both 3′-end cleavage and polyadenylation of premessenger RNA (pre-mRNA) as well as to promote downstream transcriptional termination, and b) additional elements upstream and downstream of AAUAAA that control the efficiency of utilization of AAUAAA as a poly(A) signal. There is considerable variability in these motifs in mammalian genes.

In one embodiment, optionally in combination with one or more features of the various embodiments described above or below, the polyadenylation signal sequence of the nucleic acid construct of the invention is a polyadenylation signal sequence of a mammalian gene or a viral gene. Suitable polyadenylation signals include, among others, a SV40 early polyadenylation signal, a SV40 late polyadenylation signal, a HSV thymidine kinase polyadenylation signal, a protamine gene polyadenylation signal, an adenovirus 5 EIb polyadenylation signal, a growth hormone polyadenylation signal, a PBGD polyadenylation signal, in silico designed polyadenylation signal (synthetic) and the like.

In a particular embodiment, the polyadenylation signal sequence of the nucleic acid construct is a polyadenylation signal sequence based on bovine growth hormone gene.

In specific embodiments, the nucleic acid construct according to the present invention includes a hybrid SYN1 promoter operably linked to a nucleotide sequence encoding a cPLA2e of SEQ ID NO:1 or 3, and the polyadenylation signal sequence of bovine growth hormone gene; in a preferred embodiment nucleotide sequence encoding said cPLA2e is SEQ ID NO:2 or 4.

Nucleic Acid Construct with mRNA Structure

In some embodiments the nucleic acid construct of the invention is an RNA. In a particular aspect, the nucleic acid construct has the structure of an mRNA. In a particular aspect, the mRNA can be modified. Modifications of mRNA nucleic acids are described in further detail in U.S. Patent Publication Nos. US20140206752 and US20150086614 and US20160304552 and PCT Publication Nos. WO2016011226, WO2016014846, and WO2016011306. Among others, chemically modified nucleobases, sugars, backbones, or any combination thereof, a patterned untranslated region (UTR), a microRNA (miRNA) binding site(s), etc.

Thus, in some embodiments the nucleic acid construct comprising a nucleotide sequence encoding a cPLA2e can further comprise at least one of the following features: a) a 5′cap structure; b) a 5′UTR; or c) a 3′UTR. In one aspect, the polynucleotide further comprises two of the features. In one aspect, the polynucleotide can further comprise all three of these features. The UTRs can be homologous or heterologous to the nucleotide sequence encoding the cPLA2e.

Untranslated regions (UTRs) are nucleic acid sections of a polynucleotide before a start codon (5′UTR) and after a stop codon (3′UTR) that are not translated. In some embodiments, a nucleic acid construct of the invention comprising a nucleotide sequence encoding a cPLA2e further comprises UTR (e.g., a 5′UTR or functional fragment thereof, a 3′UTR or functional fragment thereof, or a combination thereof).

In some embodiments, the nucleic acid construct comprises two or more 5′UTRs or functional fragments thereof, each of which has the same or different nucleotide sequence. In some embodiments, the nucleic acid construct comprises two or more 3′UTRs or functional fragments thereof, each of which has the same or different nucleotide sequence. In some embodiments, the 5′UTR or functional fragment thereof, 3′UTR or functional fragment thereof, or any combination thereof is sequence optimized. In some embodiments, the 5′UTR or functional fragment thereof, 3′UTR or functional fragment thereof, or any combination thereof comprises at least one chemically modified nucleobase, e.g., 1-methylpseudouridine or 5-methoxyuracil. In some embodiments, a functional fragment of a 5′UTR or 3′UTR comprises one or more regulatory features of a full length 5′ or 3′UTR, respectively.

By engineering the features typically found in abundantly expressed genes of specific target cells/tissues/organs, one can enhance the stability and protein production of a polynucleotide in that particular target cell/tissue/organ.

In some embodiments, the 5′UTR and the 3′UTR can be heterologous. In some embodiments, the 5′UTR can be derived from a different species than the 3′UTR.

Publ. No. WO/2014/164253, incorporated herein by reference in its entirety, provides a listing of exemplary UTRs that can be utilized in the polynucleotide of the present invention as flanking regions to the nucleotide sequence encoding the cPLA2e.

Wild-type UTRs derived from any gene or mRNA can be incorporated into the nucleic acid construct of the invention. In some embodiments, a UTR can be altered relative to a wild type or native UTR to produce a variant UTR, e.g., by changing the orientation or location of the UTR relative to the coding nucleotide sequence; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR. Additionally, one or more synthetic UTRs can be used in combination with one or more non-synthetic UTRs. See, e.g., Mandal and Rossi, Nat. Protoc. 2013 8(3):568-82, and sequences available at www.addgene.org/Derrick_Rossi/, the contents of each are incorporated herein by reference in their entirety.

The 5′cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing. Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or ante-terminal transcribed nucleotides of the 5′end of the mRNA can optionally also be 2′-O-methylated. 5′-decapping through hydrolysis and cleavage of the guanylate cap structure can target a nucleic acid molecule, such as an mRNA molecule, for degradation.

In some embodiments, the nucleic acid construct of the present invention incorporates as 5′cap moiety or structure.

In some embodiments, the nucleic acid construct of the present invention (i.e., a polynucleotide comprising a nucleotide sequence encoding a cPLAe) further comprises a poly-A tail.

### The Vector

The nucleic acid construct of the invention may be comprised in an expression vector; thus, in an aspect the invention relates to an expression vector that comprises a nucleic acid construct of the invention.

As used herein, the term “expression vector” or “vector” refers to a nucleic acid molecule used as a vehicle to transfer genetic material, and in particular to deliver a nucleic acid into a host cell, either in vitro or in vivo. Expression vector also refers to a nucleic acid molecule capable of effecting expression of a gene (transgene) in host cells or host organisms compatible with such sequences. Expression vectors typically include at least suitable transcription regulatory sequences and optionally, 3′ transcription termination signals. Additional factors necessary or helpful in effecting expression may also be present, such as expression enhancer elements able to respond to a precise inductive signal (endogenous or chimeric transcription factors) or specific for certain cells, organs or tissues. Vectors include, but are not limited to, plasmids, phasmids, cosmids, transposable elements, viruses, and artificial chromosomes (e.g., YACs). Preferably, the vector of the invention is a vector suitable for use in gene or cell therapy, and in particular is suitable to target neuronal cells.

In some embodiments, the expression vector is a viral vector, such as vectors derived from Moloney murine leukemia virus vectors (MoMLV), MSCV, SFFV, MPSV or SNV, lentiviral vectors (e.g. derived from human immunodeficiency virus (HIV), simian immunodeficiency virus (SIV), feline immunodeficiency virus (FIV), bovine immunodeficiency virus (BIV) or equine infectious anemia virus (EIAV)), adenoviral (Ad) vectors, adeno-associated viral (AAV) vectors, simian virus 40 (SV-40) vectors, bovine papilloma virus vectors, Epstein-Barr virus, herpes virus vectors, vaccinia virus vectors, Harvey murine sarcoma virus vectors, murine mammary tumor virus vectors, Rous sarcoma virus vectors.

As is known in the art, depending on the specific viral vector considered, suitable sequences should be introduced in the vector of the invention for obtaining a functional viral vector, such as AAV ITRs for an AAV vector, or LTRs for lentiviral vectors. In a particular embodiment, optionally in combination with one or more features of the various embodiments described above or below, said vector is an AAV vector.

AAV has arisen considerable interest as a potential vector for human gene therapy. Among the favorable properties of the virus are its lack of association with any human disease, its ability to infect both dividing and non-dividing cells, and the wide range of cell lines derived from different tissues that can be infected. The AAV genome is composed of a linear, single-stranded DNA molecule which contains 4681 bases (Berns and Bohenzky, 1987, Advances in Virus Research (Academic Press, Inc.) 32:243-307). The genome includes inverted terminal repeats (ITRs) at each end, which function in cis as origins of DNA replication and as packaging signals for the virus. The ITRs are approximately 145 bp in length. The internal non-repeated portion of the genome includes two large open reading frames, known as the AAV rep and cap genes, respectively. These genes code for the viral proteins involved in replication and packaging of the virion. In particular, at least four viral proteins are synthesized from the AAV rep gene, Rep 78, Rep 68, Rep 52 and Rep 40, named according to their apparent molecular weight. The AAV cap gene encodes at least three proteins, VP1, VP2 and VP3. For a detailed description of the AAV genome, see, e.g., Muzyczka, N. 1992 Current Topics in Microbiol. and Immunol. 158:97-129.

Thus, in one embodiment, optionally in combination with one or more features of the various embodiments described above or below, the nucleic acid construct or expression vector of the invention [comprising nucleotide sequence encoding cPLA2e] further comprises a 5′ITR and a 3′ITR sequences, preferably a 5′ITR and a 3′ITR sequences of an adeno-associated virus.

As used herein the term “inverted terminal repeat (ITR)” refers to a nucleotide sequence located at the 5′-end (5′ITR) and a nucleotide sequence located at the 3′-end (3′ITR) of a virus, that contain palindromic sequences and that can fold over to form T-shaped hairpin structures that function as primers during initiation of DNA replication. They are also needed for viral genome integration into the host genome; for the rescue from the host genome; and for the encapsidation of viral nucleic acid into mature virions. The ITRs are required in cis for the vector genome replication and its packaging into the viral particles.

AAV ITRs for use in the viral vector of the invention may have a wild-type nucleotide sequence or may be altered by insertion, deletion or substitution. The serotype of the inverted terminal repeats (ITRs) of the AAV may be selected from any known human or nonhuman AAV serotype. In specific embodiments, the nucleic acid construct or viral expression vector may be carried out by using ITRs of any AAV serotype, including AAV1, AAV2, AAV3 (including types 3A and 3B), AAV4, AAV5, AAV6, AAV7, AAV8, AAV9, AAV10, AAV11, AAV12, avian AAV, bovine AAV, canine AAV, equine AAV, ovine AAV, and any other AAV serotype now known or later discovered.

In one preferred embodiment, the nucleic acid construct or expression vector further comprises a 5′ITR and a 3′ITR of an AAV of a serotype AAV2.

In other embodiments, the nucleic acid construct or expression vector of the invention can be carried out by using synthetic 5′ITR and/or 3′ITR; and also by using a 5′ITR and a 3′ITR which come from viruses of different serotype. All other viral genes required for viral vector replication can be provided in trans within the virus-producing cells (packaging cells) as described below. Therefore, their inclusion in the viral vector is optional.

In one embodiment, the nucleic acid construct or viral vector of the invention comprises a 5′ITR, a ψ packaging signal, and a 3′ITR of a virus. “ψ packaging signal” is a cis-acting nucleotide sequence of the virus genome, which in some viruses (e.g. adenoviruses, lentiviruses . . . ) is essential for the process of packaging the virus genome into the viral capsid during replication.

The construction of recombinant AAV viral particles is generally known in the art and has been described for instance in U.S. Pat. Nos. 5,173,414 and 5,139,941; WO 92/01070, WO 93/03769, Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; and Kotin, R. M. (1994) Human Gene Therapy 5:793-801.

### The Viral Particle

The nucleic acid construct or the expression vector of the invention may be packaged into a virus capsid to generate a “viral particle”, also named “viral vector particle”.

Thus, in an aspect the present invention relates to a viral particle comprising a nucleic acid construct or an expression vector of the invention.

In an aspect the invention relates to a viral particle that includes a nucleic acid construct or expression vector comprising a promoter, operably-linked to a nucleotide sequence encoding a cPLA2e; and to a viral particle that includes a nucleic acid construct or expression vector comprising a) a promoter operably-linked to a nucleotide sequence encoding a cPLA2e, b) a polyadenylation signal sequence, and c) 5′ITR and a 3′ITR; optionally in combination with one or more features of the various embodiments described above or below.

In a preferred embodiment, the viral particle of the invention is an AAV particle comprising capsid proteins of adeno-associated virus, i.e. the nucleic acid construct or the expression vector of the invention is packaged into an AAV-derived capsid to generate an “adeno-associated viral particle” or “AAV particle”. The term AAV particle encompasses any recombinant AAV particle or mutant AAV particle, genetically engineered. A recombinant AAV particle may be prepared by encapsidating the nucleic acid construct or viral expression vector including ITR(s) derived from a particular AAV serotype on a viral particle formed by natural or mutant Cap proteins corresponding to an AAV of the same or different serotype.

Proteins of the viral capsid of an adeno-associated virus include the capsid proteins VP1, VP2, and VP3. Differences among the capsid protein sequences of the various AAV serotypes result in the use of different cell surface receptors for cell entry. In combination with alternative intracellular processing pathways, this gives rise to distinct tissue tropisms for each AAV serotype.

In an embodiment, an AAV particle according to the invention may be prepared by encapsidating the viral vector of an AAV vector/genome derived from a particular AAV serotype on a viral particle formed by natural Cap proteins corresponding to an AAV of the same particular serotype. Nevertheless, several techniques have been developed to modify and improve the structural and functional properties of naturally occurring AAV viral particles (Bunning H et al. J Gene Med, 2008; 10: 717-733; Paulk et al. Mol ther. 2018; 26(1):289-303; Wang L et al. Mol Ther. 2015; 23(12):1877-87; Vercauteren et al. Mol Ther. 2016; 24(6):1042-1049; Zinn E et al., Cell Rep. 2015; 12(6):1056-68).

Thus, in another embodiment, AAV viral particles according to the invention include the nucleic acid construct comprising the nucleotide sequence encoding the cPLA2e flanked by ITR(s) of a given AAV serotype packaged, for example, into: a) a viral particle constituted of capsid proteins derived from the same or different AAV serotype [e.g. AAV2 ITRs and AAV9 capsid proteins; AAV2 ITRs and AAV TT capsid proteins; etc]; b) a mosaic viral particle constituted of a mixture of capsid proteins from different AAV serotypes or mutants [e.g. AAV2 ITRs with a capsid formed by proteins of two or multiple AAV serotypes]; c) a chimeric viral particle constituted of capsid proteins that have been truncated by domain swapping between different AAV serotypes or variants [e.g. AAV2 ITRs with AAV5 capsid proteins with AAV3 domains]; or d) a targeted viral particle engineered to display selective binding domains, enabling stringent interaction with target cell specific receptors.

In specific embodiments, examples of AAV serotype of the capsid proteins of AAV particle according to the present invention include AAV2, AAV5, AAV9, and AAV TT. In more preferred embodiments, said AAV serotype of the capsid proteins are selected from AAV9 and AAV TT serotype.

In a particular embodiment, optionally in combination with one or more features of the various embodiments described above or below, the viral particle is an AAV particle including a nucleic acid construct or expression vector that comprises 5′ITR and 3′ITR sequences from an AAV virus; preferably AAV particle comprises capsid proteins of an AAV2, AAV5, AAV9, or AAV TT serotype, more preferably of an AAV9 serotype or of an AAV TT serotype; and/or 5′ITR and 3′ITR sequences of an AAV2 serotype.

In a particular embodiment, optionally in combination with one or more features of the various embodiments described above or below, the viral particle includes a nucleic acid construct or expression vector comprising a nucleotide sequence encoding a human cPLA2e of amino acid SEQ ID NO:1 or SEQ ID NO:3 under the control of a promoter, said promoter allowing expression of said human cPLA2e in at least neurons of the hippocampus, and said viral particle is selected among viral particles that targets at least neurons of the hippocampus, typically an AAV particle including capsid proteins selected from the group consisting of AAV2, AAV5, AAV9, or AAV TT serotypes; preferably the nucleotide sequence encoding human cPLA2e is SEQ ID NO:2 or SEQ ID NO:4, and/or the promoter is a neuronal-specific promoter, more preferably a SYN1 promoter or a hybrid SYN1 promoter.

In a more particular embodiment, such recombinant AAV particle according to the present invention comprises capsid proteins of the AAV9 or AAV TT serotype and an AAV vector comprising (i) a nucleic acid construct comprising a hybrid SYN1 promoter operably linked to a nucleotide sequence SEQ ID NO:2 or 4 encoding a human cPLA2e, and (ii) AAV ITRs, such as 5′ and 3′ ITRs of AAV2, flanking said nucleic acid construct.

The skilled person will appreciate that the AAV viral particle according to the present invention may comprise capsid proteins from any AAV serotype including AAV1, AAV2, AAV3 (including types 3A and 3B), AAV4, AAV5, AAV6, AAV7, AAV8, AAV9, AAV10, AAV11, AAV12, avian AAV, bovine AAV, canine AAV, equine AAV, ovine AAV, synthetic AAV variants such as NP40, NP59, NP84 (Paulk et al. Mol ther. 2018. 26(1):289-303), LK03 (Wang L et al. Mol Ther. 2015. 23(12):1877-87), AAV3-ST (Vercauteren et al. Mol Ther. 2016.24(6):1042-1049), Anc80 (Zinn E et al., Cell Rep. 2015; 12(6):1056-68) and any other AAV serotype now known or later discovered.

### Production of Vectors and Viral Particles

Production of viral particles carrying the expression viral vector as disclosed above can be performed by means of conventional methods and protocols, which are selected taking into account the structural features chosen for the actual embodiment of expression vector and viral particle of the vector to be produced.

Briefly, viral particles can be produced in a host cell, more particularly in specific virus-producing cell (packaging cell), which is transfected with the nucleic acid construct or expression vector to be packaged, in the presence of a helper vector or virus or other DNA construct(s).

The term “packaging cells” as used herein, refers to a cell or cell line which may be transfected with a nucleic acid construct or expression vector of the invention and provides in trans all the missing functions which are required for the complete replication and packaging of a viral vector. Typically, the packaging cells express in a constitutive or inducible manner one or more of said missing viral functions. Said packaging cells can be adherent or suspension cells.

Typically, a process of producing viral particles comprises the following steps: a) culturing a packaging cell comprising a nucleic acid construct or expression vector as described above in a culture medium; and b) harvesting the viral particles from the cell culture supernatant and/or inside the cells.

Conventional methods can be used to produce AAV viral particles which consist on transient cell co-transfection with nucleic acid construct or expression vector (e.g. a plasmid) carrying the transgene of the invention; a nucleic acid construct (e.g., an AAV helper plasmid) that encodes rep and cap genes, but does not carry ITR sequences; and with a third nucleic acid construct (e.g., a plasmid) providing the adenoviral functions necessary for AAV replication. Viral genes necessary for AAV replication are referred herein as viral helper genes. Typically, said genes necessary for AAV replication are adenoviral helper genes, such as ElA, E1B, E2a, E4, or VA RNAs. Preferably, the adenoviral helper genes are of the Ad5 or Ad2 serotype.

Large-scale production of AAV particles according to the disclosure can also be carried out for example by infection of insect cells with a combination of recombinant baculoviruses (Urabe et al. Hum. Gene Ther. 2002; 13: 1935-1943). SF9 cells are co-infected with two or three baculovirus vectors respectively expressing AAV rep, AAV cap and the AAV vector to be packaged. The recombinant baculovirus vectors will provide the viral helper gene functions required for virus replication and/or packaging. Smith et al 2009 (Molecular Therapy, vol. 17, no. 11, pp 1888-1896) further describes a dual baculovirus expression system for large-scale production of AAV particles in insect cells.

Suitable culture media will be known to a person skilled in the art. The ingredients that compose such media may vary depending on the type of cell to be cultured. In addition to nutrient composition, osmolarity and pH are considered important parameters of culture media. The cell growth medium comprises a number of ingredients well known by the person skilled in the art, including amino acids, vitamins, organic and inorganic salts, sources of carbohydrate, lipids, trace elements (CuSO4, FeSO4, Fe(NO3)3, ZnSO4 . . . ), each ingredient being present in an amount which supports the cultivation of a cell in vitro (i.e., survival and growth of cells). Ingredients may also include different auxiliary substances, such as buffer substances (like sodium bicarbonate, Hepes, Tris . . . ), oxidation stabilizers, stabilizers to counteract mechanical stress, protease inhibitors, animal growth factors, plant hydrolyzates, anti-clumping agents, anti-foaming agents. Characteristics and compositions of the cell growth media vary depending on the particular cellular requirements. Examples of commercially available cell growth media are: MEM (Minimum Essential Medium), BME (Basal Medium Eagle) DMEM (Dulbecco's modified Eagle's Medium), Iscoves DMEM (Iscove's modification of Dulbecco's Medium), GMEM, RPMI 1640, Leibovitz L-15, McCoy's, Medium 199, Ham (Ham's Media) F10 and derivatives, Ham F12, DMEM/F12, etc.

Further guidance for the construction and production of viral vectors for use according to the disclosure can be found in Viral Vectors for Gene Therapy, Methods and Protocols. Series: Methods in Molecular Biology, Vol. 737. Merten and Al-Rubeai (Eds.); 2011 Humana Press (Springer); Gene Therapy. M. Giacca. 2010 Springer-Verlag; Heilbronn R. and Weger S. Viral Vectors for Gene Transfer: Current Status of Gene Therapeutics. In: Drug Delivery, Handbook of Experimental Pharmacology 197; M. Schafer-Korting (Ed.). 2010 Springer-Verlag; pp. 143-170; Adeno-Associated Virus: Methods and Protocols. R. O. Snyder and P. Moulllier (Eds). 2011 Humana Press (Springer); Bunning H. et al. Recent developments in adeno-associated virus technology. J. Gene Med. 2008; 10:717-733; Adenovirus: Methods and Protocols. M. Chillón and A. Bosch (Eds.); Third Edition. 2014 Humana Press (Springer).

In another aspect, the invention relates to a host cell comprising a nucleic acid construct or an expression vector of the invention.

In an embodiment, the host cell according to the invention is a specific virus-producing cell, also named packaging cell, which is transfected with the nucleic acid construct or expression vector according to the invention, in the presence of a helper vector or virus or other DNA constructs and provides in trans all the missing functions which are required for the complete replication and packaging of a viral particle. Said packaging cells can be adherent or suspension cells

For example, said packaging cells may be eukaryotic cells such as mammalian cells, including simian, human, dog and rodent cells. Examples of human cells are PER.C6 cells (WO01/38362), MRC-5 (ATCC CCL-171), WI-38 (ATCC CCL-75), HEK-293 cells (ATCC CRL-1573), HeLa cells (ATCC CCL2) and fetal rhesus lung cells (ATCC CL-160). Examples of non-human primate cells are Vero cells (ATCC CCL81), COS-1 cells (ATCC CRL-1650) or COS-7 cells (ATCC CRL-1651). Examples of dog cells are MDCK cells (ATCC CCL-34). Examples of rodent cells are hamster cells, such as BHK21-F, HKCC cells, or CHO cells.

As an alternative to mammalian sources, the packaging cells for producing the viral particles may be derived from avian sources such as chicken, duck, goose, quail or pheasant. Examples of avian cell lines include avian embryonic stem cells (WO01/85938 and WO03/076601), immortalized duck retina cells (WO2005/042728), and avian embryonic stem cell derived cells, including chicken cells (WO2006/108846) or duck cells, such as EB66 cell line (WO2008/129058 & WO2008/142124).

In another embodiment, the cells can be any cells permissive for baculovirus infection and replication packaging cells. In a particular embodiment, said cells are insect cells, such as SF9 cells (ATCC CRL-1711), Sf21 cells (IPLB-Sf21), MG1 cells (BTI-TN-MG1) or High Five™ cells (BTI-TN-5B1-4).

Accordingly, in a particular embodiment, the host cell comprises: a nucleic acid construct or expression vector comprising a nucleotide sequence encoding a cPLA2e according to the invention (e.g., the AAV vector according to the invention); a nucleic acid construct, for example a plasmid, encoding AAV rep and/or cap genes which does not carry the ITR sequences; and/or a nucleic acid construct, for example a plasmid or virus, comprising viral helper genes.

In another aspect, the invention relates to a host cell transduced with an expression vector or viral particle of the invention and the term “host cell” as used herein refers to any cell line that is susceptible to infection by a virus of interest, and amenable to culture in vitro.

In other embodiments, host cell of the invention can be used for therapeutic purposes, e.g. for the therapeutic uses disclosed herein.

### Pharmaceutical Composition

Another aspect of the present invention is a pharmaceutical composition comprising a nucleic acid construct as mentioned above, a vector as mentioned above, a host cell as mentioned above, or a viral particle as mentioned above, in combination with one or more pharmaceutical acceptable excipients.

In another aspect, the invention also refers to a pharmaceutical composition comprising a cPLA2e inducing agent in any of the embodiments disclosed above or below, for use or administration in the treatment of a cognitive disorder and/or a disease associated with a cognitive disorder.

As used herein, the term “pharmaceutically acceptable” means approved by a regulatory agency or recognized pharmacopeia such as European Pharmacopeia, for use in animals and/or humans. The term “excipient” refers to a diluent, adjuvant, carrier, or vehicle with which the therapeutic agent is administered.

The pharmaceutical composition or medicament of the invention typically comprises the therapeutic agent (e.g. a vector or viral particle of the invention) in an effective amount, sufficient to provide a desired therapeutic effect, and a pharmaceutically acceptable carrier or excipient.

In a preferred embodiment, optionally in combination with one or more features of the various embodiments described above or below, the invention relates then to a pharmaceutical composition that comprises a vector or viral particle as disclosed above, and a pharmaceutically acceptable carrier.

Any suitable pharmaceutically acceptable carrier or excipient can be used in the preparation of a pharmaceutical composition (See e.g. Remington: The Science and Practice of Pharmacy, Alfonso R. Gennaro (Editor) Mack Publishing Company, April 1997). Pharmaceutical compositions are typically sterile and stable under the conditions of manufacture and storage. Pharmaceutical compositions may be formulated as solutions (e.g. saline, dextrose solution, or buffered solution, or other pharmaceutically acceptable sterile fluids), microemulsions, liposomes, or other ordered structure suitable to accommodate a high product concentration (e.g. microparticles or nanoparticles). The carrier may be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Preferably, said pharmaceutical composition is formulated as a solution, more preferably as an optionally buffered saline solution.

Preferably, said pharmaceutical composition is formulated as a solution, more preferably as an optionally buffered saline solution. Supplementary active compounds can also be incorporated into the pharmaceutical compositions of the invention. Guidance on co-administration of additional therapeutics can for example be found in the Compendium of Pharmaceutical and Specialties (CPS) of the Canadian Pharmacists Association.

In one embodiment, the pharmaceutical composition is a composition suitable for intraparenchymal, intracerebral, intravenous, or intrathecal administration. These pharmaceutical compositions are exemplary only and do not limit the pharmaceutical compositions suitable for other parenteral and non-parenteral administration routes. The pharmaceutical compositions described herein can be packaged in single unit dosage or in multidosage forms.

### Therapeutic Uses

Using an animal model of Alzheimer's disease (APP/PS1 mice) and elderly WT mice, the inventors surprisingly found that AAV-mediated enhancement of cPLA2e expression, ameliorated memory impairment in APP/PS1 mice and improved memory function in aged-WT animals.

These results provide strong evidence of a possible therapeutic strategy for the treatment of cognitive disorders and/or diseases associated with cognitive disorder in a subject, and more specifically for the treatment of dementias, e.g. of an age-related dementia or an Alzheimer's disease.

Hence, another aspect of the invention relates to a method for treating a cognitive disorder and/or a disease associated with a cognitive disorder, for example dementia, in particular age-related dementia or Alzheimer's disease, in a subject in need thereof, said method comprising administering to said subject a therapeutically effective amount of a cPLA2e inducing agent.

In a further aspect, the invention relates to a cPLA2e inducing agent, for use as a medicament in a subject in need thereof, and more specifically, for use in the treatment of a cognitive disorder and/or disease associated with a cognitive disorder, for example dementia, and more specifically age-related dementia or Alzheimer's disease, in a subject in need thereof.

In a related aspect, the invention pertains to the use of a cPLA2e inducing agent for the manufacturing of a medicament, more specifically, for the treatment of a cognitive disorder and/or disease associated with a cognitive disorder, for example dementia, and more specifically age-related dementia or Alzheimer's disease.

The cPLA2e inducing agent for use or administration in the treatment of cognitive disorders and diseases associated with a cognitive disorder according to the invention, can be selected, among others, from the group consisting of: a) a nucleic acid construct of the invention, as mentioned above; b) a vector comprising a nucleic acid construct of the invention, as mentioned above; c) a viral particle comprising a nucleic acid construct or vector of the invention, as mentioned above; d) a host cell comprising a nucleic acid construct or vector according to the invention; e) a cPLA2e polypeptide or protein; and f) a pharmaceutical composition comprising any of said nucleic acid construct, vector comprising the nucleic acid construct, viral particle comprising the nucleic acid construct or vector, and cPLA2e polypeptide or protein.

Regarding said cPLA2e polypeptide or protein, any and all the embodiments and preferred embodiments mentioned above for the cPLA2e encoded by the nucleotide sequence included in the nucleic acid construct of the invention are embodiments within the scope of the cPLA2e polypeptide or protein for use or administration in the treatment according to the invention; in particular, a cPLA2e comprising or consisting of amino acid SEQ ID NO:1 or 3 or a variant with at least 70% sequence identity thereto; optionally as a fusion protein with another polypeptide(s), such as a tag or carrier polypeptide.

In a preferred embodiment, the cPLA2e inducing agent for the therapeutic use of the invention is a vector of the invention, more preferably a viral vector, or a viral particle (e.g. an AAV particle), or the pharmaceutical composition that comprises it.

As used herein, the terms “cognitive disorder” and “cognitive impairment,” interchangeably refer to any impairment of cognition such as a condition characterized by one or more of the following behaviors: inhibition of at least one form of learning (e.g., associative learning), inhibition of at least one form of memory function (e.g., executive function), inhibition of learning, inhibition of memory acquisition, inhibition of memory recall, suppression of long term potentiation (LTP) in the hippocampus, or a combination thereof. In humans, any suitable method for testing and neuroimaging of the hippocampus or brain function can be used to determine or assess cognition and its potential impairment. For example, cognition (e.g., memory or learning) and its potential impairment can be measured using any suitable psychological test, including without limitation, “Kiel Locomotor Maze”, containing features of the Radial Arm Maze and the Morris Water Maze, in order to assess spatial memory and orientation, which has been optimized for school age children, or Cambridge Neuropsychological Test Automated Battery (CANTAB), a computerized battery of nonverbal visually-presented neuropsychological tests, designed to test spatial memory span, spatial working memory and spatial recognition. Additionally, the outcome of methods related to cognitive impairment disclosed herein can be shown via comparative testing in animals (e.g., rats or mice), using the same composition administered to humans.

The cPLA2e inducing agents of the invention are particularly useful for the treatment of cognitive disorders associated to conditions which impair or otherwise affect normal functioning of the central nervous system and diseases associated with cognitive disorders, such as dementias (e.g. age-associated dementia (senile dementia), cerebrovascular dementia, and/or neurodegenerative dementing disease with aberrant protein aggregations as specially Alzheimer's disease, Parkinson disease, ALS, or prion diseases, as Creutzfeldt-Jakob disease or Gerstmann-Straussler-Scheinher disease); mild cognitive impairment, attention deficit disorder, among others. Thus, the cPLA2e inducing agents of the invention are used in particular for the treatment of cognitive disorders associated to one of these conditions.

In a preferred embodiment, the cPLA2e inducing agent is particularly useful for the treatment of a cognitive disorder in a patient with Alzheimer's disease. As used herein, Alzheimer's disease (AD) refers to a progressive neurodegenerative disorder of the central nervous system of an unknown origin. Within the context of AD, the defining characteristic is cognitive impairment. AD is defined as a neurodegenerative disorder that constitutes the main common form of dementia in the elderly: it is characterized by accumulation of two abnormal proteins in the brain: beta-amyloid peptide and hyperphosphorylated tau in form of amyloid plaques and neurofibrillary tangles respectively. The criteria issued in 1984 by the National Institute of Neurological and Communicative Disorders and Stroke (NINCDS) and the Alzheimer's disease and Related Disorders Association (ARRDA) for AD diagnosis include: (1) two or more areas involved, (2) presence of progressive dementia, (3) absence alteration of consciousness, (4) beginning between 40 and 90 years and (5) cannot be explained by other cause. Distinctive and reliable biomarkers of AD are now available through structural MRI, molecular neuroimaging with PET, and cerebrospinal fluid analyses to confirm AD diagnosis.

Furthermore, the prodromal pre-Alzheimer's disease state known as Mild Cognitive Impairment (MCI) is defined as an objective abnormal memory loss for the age and educational level of a subject. Criteria for MCI include: (1) Memory complaints corroborated by a family member, (2) other cognitive functions are normal, (3) normal daily activities, (4) abnormal memory for the age and (5) absence of dementia.

The term “subject” or “patient” as used herein, refers to mammals. Mammalian species that can benefit from the disclosed methods of treatment include, but are not limited to, humans, non-human primates such as apes, chimpanzees, monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like.

As used herein, “treatment”, “treating” or “treat” refer to: (i) preventing or retarding a disease, disorder or condition from occurring in a subject which may be predisposed to the disease, disorder and/or condition but has not yet been diagnosed as having it; (ii) inhibiting the disease, disorder or condition, i.e., arresting or slowing down its development or progression; and/or (iii) relieving the disease, disorder or condition, i.e., causing regression of the disease, disorder and/or condition. In certain embodiments, such term refers to the amelioration or eradication of a disease or symptoms associated with a disease.

In relation to cognitive impairment, “treatment”, “treating” or “treat” refer to: (i) preventing or retarding cognitive impairment from occurring in a subject which may be predisposed to cognitive impairment but has not yet been diagnosed as having it; (ii) inhibiting cognitive impairment, i.e., arresting or slowing down its development or progression; (iii) relieving cognitive impairment, i.e., causing its regression; and/or (iv) enhancing cognitive performance. The term “treating cognitive impairment,” unless otherwise indicated herein, refers to reducing cognitive impairment, ameliorating at least one symptom relating to or resulting from cognitive impairment (such as a symptom of a disease or disorder that could cause cognitive impairment), or both. The treatment of cognitive impairment relates, in particular, to the treatment of learning and memory impairments, and to the enhancement of learning and memory performance. “Enhancing learning and memory performance” refers to improving or increasing the mental faculty by which to register, retain or recall past experiences, knowledge, ideas, sensations, thoughts or impressions.

As used herein a “therapeutically effective amount” refers to an amount effective, at dosages and for periods of time necessary to achieve the desired therapeutic result, such as one or more of the following therapeutic results: a significant delay of the onset or progression of the disease; a significant reduction of the severity of one or more symptoms; a significant reduction of hallmarks of AD, amyloid and/or tau pathology; a significant increase in synaptic plasticity; a significant attenuation of mortality associated to aging and/or AD.

A therapeutically effective amount is also typically one in which any toxic or detrimental effect of the product or pharmaceutical composition is outweighed by the therapeutically beneficial effects.

In one embodiment the nucleic acid construct, expression vector, viral particle, host cell, cPLA2e polypeptide or protein or pharmaceutical composition for its therapeutic use according to the invention is administered to the subject or patient by a parenteral route, e.g. by intraparenchymal, intracerebral, intracerebroventricular (icv), intrathecal, intranasal, intravenous, or subcutaneous route.

Typically, a therapeutically effective amount of said nucleic acid construct, expression vector, viral particle, host cell, cPLA2e polypeptide or protein, or pharmaceutical composition is preferably administered by intrathecal or intraparenchymal route, the latter preferably to brain areas such as the hippocampal formation or cerebral cortex. The intraparenchymal route may facilitate preferred local administration to hippocampus and cortex as compared to other area of the brain. As used herein, a “preferred local administration to hippocampus” does not mean that all the cPLA2e inducing agent is administered to said areas of the brain, but a majority, for example at least 50%, at least 60%, at least 70%, or at least 80% of the cPLA2e inducing agent is administered to said areas.

The therapeutically effective amount of the cPLA2e inducing agent (e.g. a nucleic acid construct, expression vector, viral particle, host cell, or cPLA2e polypeptide or protein), or the pharmaceutical composition that comprises it, may vary according to factors such as the disease state, age, sex, and weight of the individual, and the ability of the product or pharmaceutical composition to elicit a desired response in the individual. Dosage regimens may be adjusted to provide the optimum therapeutic response.

For any particular subject, specific dosage regimens may be adjusted over time according to the individual needs and the professional judgment of the person administering or supervising the administration of the compositions. Dosage ranges set forth herein are exemplary only and do not limit the dosage ranges that may be selected by medical practitioners.

In one embodiment, an AAV viral particle according to the invention can be administered to the human subject or patient for the treatment of a cognitive disorder or a disease associated with a cognitive disorder, such as Alzheimer's disease, in an amount or dose comprised within a range of 10⁸ to 10¹⁴ vg/kg (vg: viral genomes; kg: subject's or patient's body weight), for example 1×10¹⁰ to 5×10¹⁴ vg/kg. In a more particular embodiment, an amount comprised within a range of 1×10¹² to 1×10¹³ vg/kg is administered. In an alternative embodiment, an amount or dose comprised within a range of 1×10⁹ to 1×10¹¹ iu/kg (iu: infective units of the vector) is administered.

In another aspect, the invention further relates to a kit comprising a nucleic acid construct, expression vector, host cell, viral particle of the invention, or a pharmaceutical composition comprising said nucleic acid construct, vector, host cell or viral particle, in one or more containers. The kit may include instructions or packaging materials that describe how to administer the nucleic acid construct, expression vector, viral particle, host cell or pharmaceutical compositions contained within the kit to a patient. Containers of the kit can be of any suitable material, e.g., glass, plastic, metal, etc., and of any suitable size, shape, or configuration. In certain embodiments, the kits may include one or more ampoules or syringes that contain the products of the invention in a suitable liquid or solution form.

The following examples are provided by way of illustration, and they are not intended to be limiting of the present invention. Furthermore, the present invention covers all possible combinations of particular and preferred embodiments described herein.

Method of Screening for New Agents Useful in the Treatment of a Cognitive Disorder and/or Disease Associated with a Cognitive Disorder.

The inventors have also put their efforts on developing systems to screen compound candidates, such as a peptide, polypeptide (e.g., antibodies) or small molecule candidates, taking advantage of the fact that cPLA2e induction or increase results in a very clear increase of the calcium-dependent N-acyltransferase (Ca-NAT) activity.

Therefore, the present invention also provides a method for identifying a compound as a candidate for the treatment of a cognitive disorder and/or disease associated with a cognitive disorder which comprises the steps of:
a) contacting the compound with mammalian assay cells;
b) checking whether an effect related to cPLA2e induction or increase is produced;
c) identifying the compound as a candidate for the treatment of a cognitive disorder and/or disease associated with a cognitive disorder if such effect is produced compared with a control.

A possible embodiment of the method of the invention is carrying out an in vitro method, wherein the assayed cells are mammalian cells, such as HEK293T cells. These cells are cultured in a medium suitable for cell growth and proliferation in the presence of the candidate compound (e.g. for 30 minutes or 1 hour), with or without ionomycin (e.g. 2 μm) and Ca-NAT activity is tested (e.g. by targeted metabolite profiling) with regard to a control that has had no contact with the candidate compound. If Ca-NAT activity is found to be increased with respect to that of control cells, the compound is identified as a possible candidate for the treatment of a cognitive disorder and/or disease associated with a cognitive disorder.

In some embodiments, cPLA2e-transfected cells (e.g., with a nucleic acid construct of the invention) could be used as control positive of cPLA2e activity induction.

The term “induce or increase” as used herein may refer to the ability to cause an overall increase, preferably of 20% or greater, more preferably of 50% or greater, and most preferably of 60%, 65%, 70%, 75%, 80%, 85%, 90%, 95%, or greater.

### EXAMPLES

To determine whether PLA2G4E has a role in learning and memory functions, the inventors overexpressed PLA2G4E in the brain of a) APP/PS1 mice (model for AD) and b) (aged—wild type animals), which are both affected with cognitive impairment. To that purpose, they constructed an AAV vector carrying as transgene Mus musculus cPLA2e and administered it to the animals. Learning and memory functions were then assessed by the MWM method.

### Example 1. Preparation of AAV2/9-mPLA2G4E

AAV2/9-mPLA2G4E vector was constructed, which includes as transgene the nucleotide sequence SEQ ID NO:5 encoding a murine PLA2G4E fused to a flag sequence through a linker.

Firstly, a 3108 bp fragment, containing the mouse PLA2G4E fused to FLAG sequence, was excised from the plasmid pRK5-PLA2G4E (a gift by B. J Cravatt; disclosed in Ogura Y et al. Nat. Chem. Biol. 2016; 12(9): 669-671) by digestion with XmnI and SacI, both in CutSmart® Buffer; separated by electrophoresis en 1% agarose gel; and then extracted from the gel by QIAquick® Gel Extraction kit (QUIAGEN) and purified through QIAquick® PCR Purification kit (QUIAGEN).

Secondly, a 4163 bp backbone fragment was obtained from plasmid pAAV-ha-synucleinA53T (kindly gifted by Dr. J. Gerez), by running a digestion with Xhol (in CutSmart® Buffer) and a subsequent treatment with Klenow polymerase, dNTPs and NEB2.1 buffer. After purification, the 4163 backbone fragment was then digested with SacI (in CutSmart® Buffer) and dephosphorylated using the Shrimp Alkaline Phosphatase rSAP (New England Biolabs, MA, USA; Weissig, H. et al. Biochem. J. 1993; 290: 503-508) to avoid vector re-ligation. The 4163 pb backbone fragment was finally separated, extracted and purified as described above for 3108 bp fragment.

Finally, in order to obtain plasmid pAAV2-mPLA2G4E, the 3108 bp fragment was cloned into the 4163 bp backbone fragment by means of a treatment with a T4 DNA ligase (Invitrogen).

Once the pAAV2-mPLA2G4E with desired construct was produced, it was then subjected to several amplification steps to generate an appropriate amount of plasmid for final virus production. Initially, E. coli chemically-competent bacteria were transformed with the plasmid using TOP10 electro-competent cells (Invitrogen) and the bacteria that had incorporated the plasmid were selected by plating on LB medium with ampicillin (50 g/ml). Then, the plasmid was obtained and purified from the bacteria using a QIAprep® Spin Miniprep kit (QUIAGEN). After checking the presence and correct orientation of the insert as well as the presence of the AAV2 ITRs, a commercial QIAGEN® Plasmid Maxi kit (QUIAGEN) was used to obtain and purify the desired amount of plasmid from the ampicillin resistant clones.

Once the vector plasmid had been constructed and purified, the AAV vector particles were produced by double transfection into HEK-293T cells of the plasmid pAAV2-mPLA2G4E and of a pDP9 helper plasmid that expresses adenoviral molecules required for production and packaging of the AAV: AAV9 cap and AAV2 rep (Durocher, Y., S. Perret, and A. Kamen., 2002, Nucleic Acids Research, 30(2):9e-9).

The vector particles were finally purified by iodixanol gradient and titrated by quantitative PCR. Viral titration, expressed as viral particles (vp)/ml, was obtained through quantitative PCR (q-PCR) using primers for mouse PLA2G4E,

| | forward primer: |
|---|---|
| (SEQ ID NO: 6) | |
| | ATGGTGACAGACTCCTTCGAG; |
| | and |
| | reverse primer: |
| (SEQ ID NO: 7) | |
| | CCTCTGCGTAAAGCTGTGG. |

The viral title obtained was 2.6×10¹¹ vp/ml.

### Example 2. General Methods

### Mice

APP/PS1 mice. The murine APP/PS1 model expresses human transgenes for the amyloid precursor protein (APP) bearing the Swedish mutation (K595N/M596L) and for the PSEN1 containing a L166P mutation, both driven by the Thy1 promoter. These mice are on an inbred C57BL/6J genetic background. The AD murine model APP/PS1 is a more accelerated amyloidosis model than the Tg2576. In these mice, the expression of human APP transgene is approximately three times higher than that of endogenous murine APP and human A042 is preferentially generated over A040. Moreover, amyloid plaque deposition starts in the hippocampus at 3-4 months (Radde et al., 2006, EMBO reports; 7(9):940-946)(Maia L F et al., 2013, Science translational medicine; 5(194):94-194) and cognitive impairment is presented from 7 months (Serneels L et al., 2009, Science; 324(5927):639-642). In the case of APP/PS1 and their correspondent negative littermates, both male and female 16-19 months old mice were used.

Aged wild type mice. Wild type mice present age-related memory deficits. Specifically, in the Morris water maze, aged wild type mice do not form a robust memory of the platform location in the hidden phase because they perform significantly worse than young mice during probe trials. These mice were on an inbred C57BL/6/SJL genetic background.

Two months-old male wild-type (WT) C57BL/6 mice, were also used to test the implication of PLA2G4E on synaptic activity.

### Stereotactic Surgery for Viral Administration

To overexpress PLA2G4E in hippocampal neurons, mice were administered with AAV2/9-mPLA2G4E in the CA1 region of the hippocampus through a stereotactic surgery. This procedure is based on a three-dimensional system of axes and spatial coordinates that allows the localization of specific points in the mouse brain (given as three-dimensional distances in millimeters (mm)) taking bregma or lambda, two easily identifiable points in the brain, as reference. The coordinates chosen for hippocampal CA1 injection using as reference the mouse atlas (G. Paxinos and K. B. J. Franklin, The mouse brain in stereotaxic coordinates, Academic Press, 1997), were: antero-posterior −2.0 mm; half-side±1.7 mm; back-ventral −2.0 mm using bregma point (formed by the intersection between the sagittal and coronal suture). Before viral administration or sham-procedure (only the surgery, without injecting anything), animals were anesthetized with an intraperitoneal (i.p.) dose of 80/10 mg/Kg of ketamine/xylazine and treated with the analgesic buprenorphine (Buprex®) at a dose of 0.1 mg/Kg. Once they were fully anesthetized, mice were placed in the stereotactic device with the head completely immobilized. After disinfecting the area with 960 alcohol, an antero-posterior cut was made in the skin using a scalpel, releasing thus the skull from its periosteum and leaving visible the bregma and lambda reference points. Next, with the help on a drill bill, a hole was made in the skull and a 5 μl Hamilton syringe was placed on the stereotaxic arm loaded with the vector viral particles (2.6×10E8 genomic copies) or unloaded (for sham procedure). Once positioned on the exact coordinate, 1 μl of the solution was injected at 0.2 μl/min and then, the syringe was maintained there for another 2 min to allow correct diffusion of the virus before withdrawing slowly the syringe. For sham-injected mice, syringe remained in the brain 5 min before the withdrawing. The same procedure was repeated for the other hemisphere. Once animals were bilaterally injected, the wound was sutured and povidone iodine (Betadine®) was administered topically. Then, animals were placed on an electric blanket to avoid heat lost until their awakening. Finally, they were stabled individually in clean cages, with easy access to softened-in water food to facilitate food intake after surgery. Throughout the intervention, physiological serum was continuously applied in mice eyes to avoid their dryness and consequent loss of vision.

MWM Test

Spatial memory was tested using the Morris Water Maze test, that analyzes both spatial and working memory so it is considered as a consistent test for hippocampal damage evaluation, which is one of the main characteristics of AD in humans (D'Hooge and Deyn, 2001, Brain research reviews; 36(1):60-90).

This test is carried out in a circular pool (diameter 1.2 m) filled with water at 20° C. and made opaque by the addition of non-toxic white paint. The pool is divided into four imaginary quadrants, in one of which is located the platform that the mouse must learn to locate in order to escape from water and be safe. In each of the four walls that surround the pool there is a picture of a geometric figure that would serve as a guide for the mouse and that would be cover or uncover depending on the phase of the test. Throughout the test, mice behavior was monitored by a camera anchored in the ceiling, just above the pool, and recorded with an HVS system to allow the subsequent analysis of escape latencies, swimming speed, path length and percentage of time spent in each quadrant of the pool using the software SMART-LD (Panlab).

Three different phases can be distinguished in the MWM test:

1) Visible-platform phase: in this phase, the platform was located in the center of one of the quadrants, elevated 1 cm above the water and identified by a piece clearly visible to the animal in order to facilitate its location. Here, mice should became familiar with the pool and learn to go to the platform to escape from water, so the visual clues remain hidden. For the visible-platform phase, mice were trained 8 times per day during 3 consecutive days. In each trial, mice had 60 s to locate the platform; if they could not reach it during this period, they were placed on it. Once the animal was on the platform, he was allowed to inspect it for 15 s before being returned to his cage.

2) Hidden-platform phase: In the second phase of the test, the platform was located in an opposite quadrant to that on the visible platform-phase. It was submerged 1 cm below the water level, without any piece above it. In this stage, mice should learn how to locate the platform with the help of the clues presented in the walls, that are now uncovered. For it, mice were trained four times per day during 7 days. As in the previous stage, mice had 60 s to reach the platform. If they could not locate it in 60 s, they were led to it. In both cases, they remained on the platform for 15 s. Three random start positions were established in each of the quadrants not occupied by the platform to avoid the appearance of trajectory preferences in mice.

3) Probe trial: memory retention was evaluated in probe trials carried out on day 6^{th} and 8^{th} of the hidden-platform phase, just before starting the hidden-platform trials of those days. For this test, the platform was removed from the pool and animals were allowed to swim for 60 s. The time that mice spend in the quadrant where the platform was placed during the hidden-platform phase is considered an estimate of memory retention degree. Retention rates higher than 25% are considered indicative of learning while those lower than 25% are considered random. Time spent in the correct quadrant was analyzed both, during the first 15 s of the test and during the whole 60 s as it has been suggested that the sensitivity of the MWM test can be increased by giving shorter probe trials (Gerlai, 2001, Behavioural Brain Research; 125(1-2):269-277).

### Dendritic Spine Density Measurement by Golgi-Cox Staining

In order to analyze dendritic spine density and morphology, a modified Golgi-Cox method was used (Glaser, Edmund M. and Hendrik Van der Loos, 1981, Journal of Neuroscience Methods 4(2):117-25). Firstly, half-brains were incubated in Golgi-Cox solution (1% potassium dichromate, 1% mercury chloride, 0.8% potassium chromate) just after being removed from the skulls for 48 h at RT and protected from light. After that time, solution was renewed and tissue was maintained there for another 3 weeks. Thereafter, brains were washed with distilled water and maintained in 900 ethanol for 30 min until they were processed in 200 m-thick coronal slices using a vibratome. Afterwards, slices were incubated in 70° ethanol, washed with distilled water, reduced in 16% ammonia for one hour and fixed in 1% sodium thiosulfate for 7 min. After another wash, slices were placed in microscope slides, dehydrated in an increasing alcohol graduation and mounted with DPX Mountant (VWR, BDH Prolabo®).

Spine density was determined in the secondary apical dendrites of the pyramidal cells located within the CA1 region of the hippocampus. Each selected neuron was captured using a Nikon Eclipse E600 light microscope and images were recorded with a digital camera (Nikon DXM 1200F) at a resolution of 1,000-1,500 dots per inch (dpi). Secondary dendrites taken between 100-200 m apart from the soma, where spine density is relatively uniform in CA1 pyramidal neurons (Megias, M., Z. Emri, T F Freund, and A I Gulyás, 2001, Neuroscience 102(3):527-40), were used for the quantification. For each mouse (n=4 per group), 3 dendrites of 9 different neurons were used for the analysis.

### Fear Conditioning Test (FC)

FC paradigm was used to analyze the effect of PLA2G4E expression on fear memory. This behavioral test consists of three phases: habituation, training and test. It was carried out in a StartFear system (Panlab). During habituation phase, mice were habituated to the conditioning chamber for 3 min with no stimuli presented. After twenty-four hours, during the training phase, they were placed again in the same chamber and were allowed to explore for 2 min. After that, they received two footshocks (0.3 mA) of 2 s separated by an interval of 30 s and were returned to their home cages after another 30 s. The following day, mice were returned to the conditioning chamber and allowed to explore the context for 2 min. Freezing behavior was recorded during this time and freezing scores were expressed as percentages. The T24 group was sacrificed 24 h after the training and the TT group 1 h after the test. The naïve group was sacrificed without performing any step of the paradigm.

### Protein Extracts

To obtain total protein extracts, brain samples were homogenized in lysis buffer with protease inhibitors (10 mM Tris-HCl pH=7.5, 1 mM NaF, 0.1 mM Na3VO4, 2% SDS), sonicated for 2 min, left 20 min on ice and centrifuged at 15,700 g for 13 min at 8° C. The supernatant was stored at −80° C. Total protein concentrations were determined using the Pierce™ BCA Protein Assay kit (Thermo Scientific).

### Immunoblotting

Protein samples were mixed with 6× Laemmli sample buffer, boiled for 5 min at 95° C., resolved onto SDS-polyacrylamide gels and transferred to nitrocellulose membranes.

Membranes were then blocked with 5% milk in TBS and incubated overnight with the following primary antibodies: rabbit polyclonal anti-pGluA1-Ser831(1:1000, Millipore), rabbit monoclonal anti-pCREB (Ser133) (1:1000, Cell Signaling), mouse monoclonal anti-synapsin I (1:1000, Synaptic Systems), rabbit polyclonal anti-PLA2G4E (1:1000, Proteintech) and mouse monoclonal anti-β-actin (1:100000, Synaptic Systems) in the corresponding buffer. After two washes in TBS/Tween-20 and one in TBS alone, immunolabeled protein bands were detected with an HRP-conjugated anti-rabbit or anti-mouse antibody (1:5000, Santa Cruz). Antibody binding was then visualized by enhanced chemiluminescence system (ECL, GE Healthcare Bioscience), and autoradiographic exposure to Hyperfilm™ ECL (GE Healthcare Bioscience). Quantity One™ software v.4.6.3 (Bio-Rad) was used for protein quantification.

### Knocking Down PLA2G4E in Primary Neuronal Culture

We used specific small interfering RNA (siRNA) to inhibit PLA2G4E expression in primary neuronal cultures. To identify effective targeting sequences for RNAi, the full-length coding sequence of murine PLA2G4E was analyzed using different algorithms. After probing a high efficacy to inhibit PLA2G4E expression, the candidate sequence was used to design a construct that carried H1 promoter operatively linked to shRNA sequence (SEQ ID NO:8; i.e. 21 base sense and antisense sequences connected with a hairpin loop (TCAAGAGA)) followed by a poly(T) termination signal. The construct with the shRNA was then cloned into an adeno-associated virus serotype 9 (AAV9-shPLA2G4E) for stable siRNA delivery (Unitat de Producció de Vectors, Barcelona).

To evaluate the selective inhibition by PLA2G4E of activity-dependent signaling, we used primary neuronal cultures as a model to study synaptic responses by evoked bursts signals in functional neural networks. Primary neuronal cultures were obtained from the hippocampus and cortex of embryonic day 16 (E16) wild type (WT) mice (A. Ricobaraza Neuropsychopharmacology, (2009); 34: 1721-1732) and infected with AAV9-shPLA2G4E or AAV9-shScrambled control on day in vitro (DIV) 1. Then, to trigger bursts of action potential firing, these cultures were treated at DIV 14 with bicuculline (50 μM, 1 h), a GABA A receptor antagonist (Arnold et al., 2005 J. Physiol. 564: 3-19) (Rao et al., Nat. Neurosci., 2006; 9: 887-895). Proteins were extracted in 2% SDS buffer and activation of CREB (phosphorylated at Ser133), pGluA1 and synapsin I expression were tested in lysates by immunoblot.

### Example 3. Effect of AAV2/9-mPLA2G4E on Memory Function of APP/PS1 Mice

A first group (n=9) of male and female 16-19 months old APP/PS1 mice were treated by stereotactic surgery with AAV2/9-mPLA2G4E as described above. Same way, a second group (n=6) of 16-19 APP/PS1 mice (sham-injected), and a third group (n=9) of non-transgenic mice (n=9) of the same ages were included as positive (with memory deficit) and negative controls (without AD-related memory impairment). Two months after the stereotactic surgery, spatial memory was tested by the MWM test as described above. Mice underwent 3 days of visible platform phase followed by 7 days of hidden-platform phase. Memory retention was tested in probe trials carried out on days 6^{th} and 8^{th}, just before starting the correspondent hidden platform-phase trial.

During the last trial of the visible-platform phase, no significant differences were observed among groups (data not shown), indicating that all animals were able to perform the task in the same conditions.

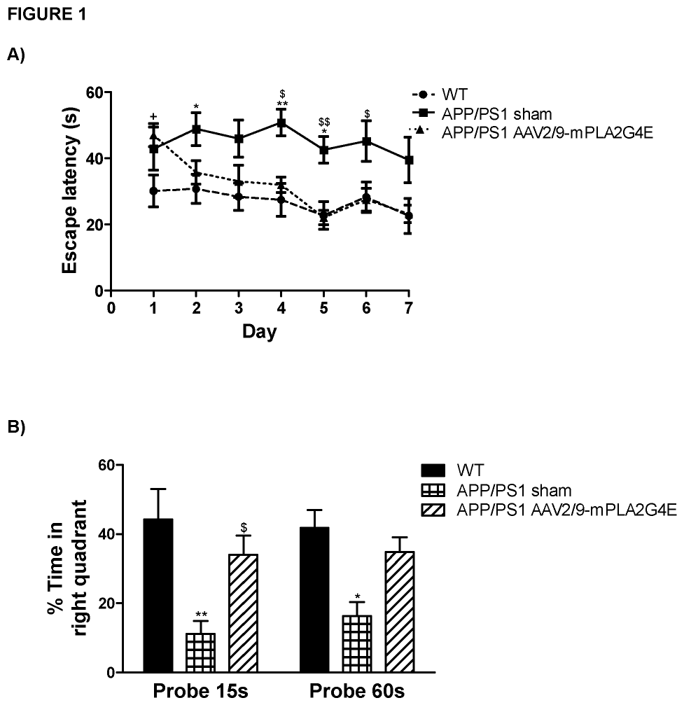

In the hidden-platform phase, as it was expected, APP/PS1 mice behaved significantly worse than WT mice, confirming the spatial memory impairment associated with this AD mouse model (FIG. 1A). Interestingly, treatment with AAV2/9-mPLA2G4E rescued spatial working-memory impairment (FIG. 1A).

Moreover, as depicted in FIG. 1B, mice treated with the AAV2/9-mPLA2G4E spent more time in the right quadrant than sham-injected mice during the probe trial on day 6^{th} Similar results were obtained in the probe trial taken on day 8^{th} (data not shown) indicating that PLA2G4E overexpression also reversed the memory retention deficits presented by APP/PS1 elderly mice.

On the other hand, a 33% of mortality was observed in sham-injected APP/PS1 mice compared to a 10% and 0% in AAV2/9-mPLA2G4E injected APP/PS1 and non-transgenic mice respectively.

In conclusion, hippocampal PLA2G4E overexpression in elderly APP/PS1 mice mediated by AAV2/9-mPLA2G4E treatment significantly rescued spatial memory impairment two months after stereotactic injection.

The Golgi-Cox method was used to analyze whether the behavioral recovery induced by PLA2G4E overexpression was reflected by structural changes in dendritic spine density. Specifically, apical dendrites from pyramidal neurons of the CA1 region of the hippocampus were studied.

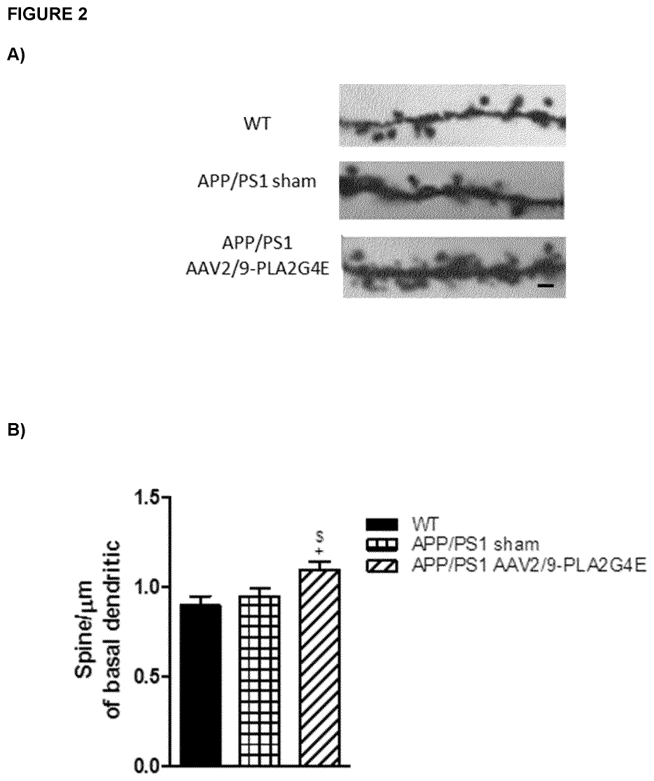

As depicted in FIGS. 2A and 2B2/9-PLA2G4E virus was able to significantly increase dendritic spine density respect to both WT, and APP/PS1 sham mice. Non-differences were found between WT and APP/PS1 sham mice.

These results suggest that changes in spine density might account for the memory recovery observed in the group of PLA2G4E overexpressing APP/PS1 mice.

### Example 4. Effect of AAV2/9-mPLA2G4E in Memory Function of Elderly Wild Type Mice

The effect of PLA2G4E overexpression on memory function in female 17 months old C57BL/6/SJL WT mice was also evaluated. A first group (n=5) of C57BL/6/SJL WT mice were treated by stereotactic surgery with AAV2/9-mPLA2G4E; a second control group (n=4) of C57BL/6/SJL WT mice were sham-injected. Three months after stereotactic surgery procedure, spatial memory was tested as described above by MWM test. In this case, the hidden-platform phase only took 6 days and probe trials were performed on days 5^{th} and 7^{th}.

Non-significant differences were observed between groups in the visible-platform phase (data not shown), indicating that all mice were able to perform the task similarly.

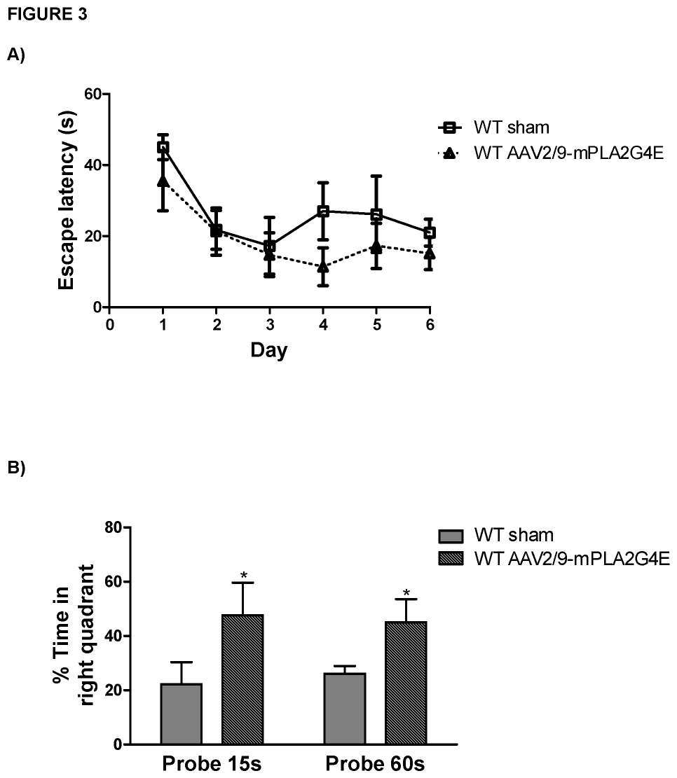

Although there were not significant differences between the two groups in the hidden-platform phase (FIG. 3A), mice treated with AAV2/9-mPLA2G4E spent more time in the right quadrant during the probe trials performed on day 5^{th} (FIG. 3B) and 7^{th} (data not shown) than sham-injected mice, indicating that viral PLA2G4E hippocampal overexpression improves memory retention rates in elderly WT mice.

In summary, hippocampal PLA2G4E overexpression mediated by AAV2/9-mPLA2G4E treatment improved memory retention in elderly C57BL/6/SJL WT mice three months after injection.

### Example 5. Role of PLA2G4E in Memory Function: Up-Regulation of PLA2G4E Expression after Fear Conditioned Memory Retrieval

To obtain more direct evidence of a functional role of PLA2G4E in learning and memory, we tested whether PLA2G4E expression was regulated in the fear conditioning (FC) test. This task requires hippocampal-dependent transcription and protein synthesis, and it has been widely used to characterize the biochemical requirements for memory formation (Huff et al., 2006 J. Neurosci., 26, pp. 1616-1623).

pCREB and PLA2G4E expression in the brain was analyzed in immunoblots after fear-memory consolidation in 2-month-old C57BL/6 J WT mice sacrificed one hour after being tested in the FC paradigm (TT group; n=8), and it was compared to that of mice sacrificed 24 h (T24 group; n=7) after the training phase of FC and to that of mice that were not subjected to any aspect of the FC test (naïve group; n=8).

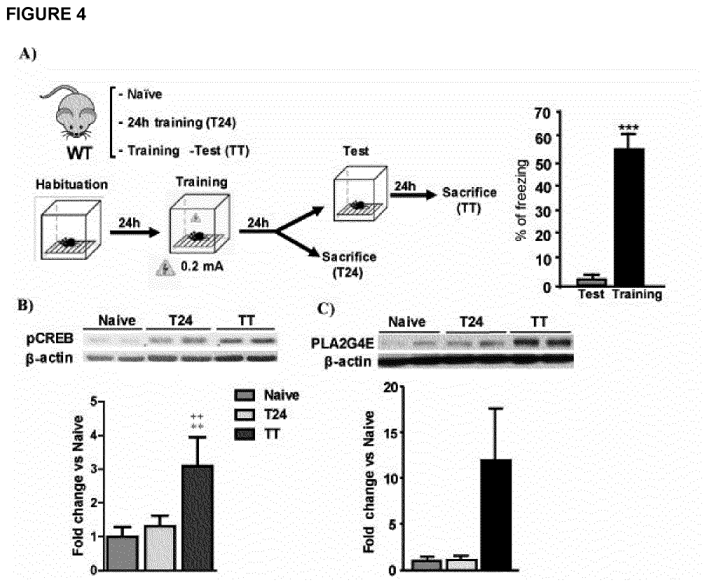

As expected, there was a significant increase (P<0.001) in freezing time (indicative of memory formation) among the mice re-introduced into the cage (TT group) during the test phase relative to that of the training phase (FIG. 4A).

As CREB-mediated transcription is necessary for consolidation and reconsolidation of contextual fear memory (Kida et al., 2002 Nat. Neurosci., 5, pp. 348-355), pCREB was first analyzed as indicative of neural plasticity in the hippocampus of the animals. An up-regulation of pCREB in the hippocampus was observed in the group of mice re-introduced into the cage.

Surprisingly, PLA2G4E expression was also stronger in both of these areas in this group of mice compare to the others (FIG. 4C).

In summary, these data suggest that there is an increase in PLA2G4E in the hippocampus during contextual memory retention, after the retrieval of a consolidated memory.

### Example 6. Role of PLA2G4E in Synaptic Plasticity: The Knockdown of PLA2G4E Blocks the Activation of Synaptic Proteins Involved in Synaptic Transmission

Considering the plausible role of PLA2G4E in memory function, in vitro assays were conducted to further characterize its role on synaptic activity.

A well-characterized protocol in cortical and hippocampal primary neurons based on the exposure to the GABA (A) receptor antagonist bicuculline (50 μM, 1 h) which is able to induce and/or increase synaptic efficacy at excitatory synapses (Rao et al., 2006 Nat. Neurosci., 9: 887-895) was used.

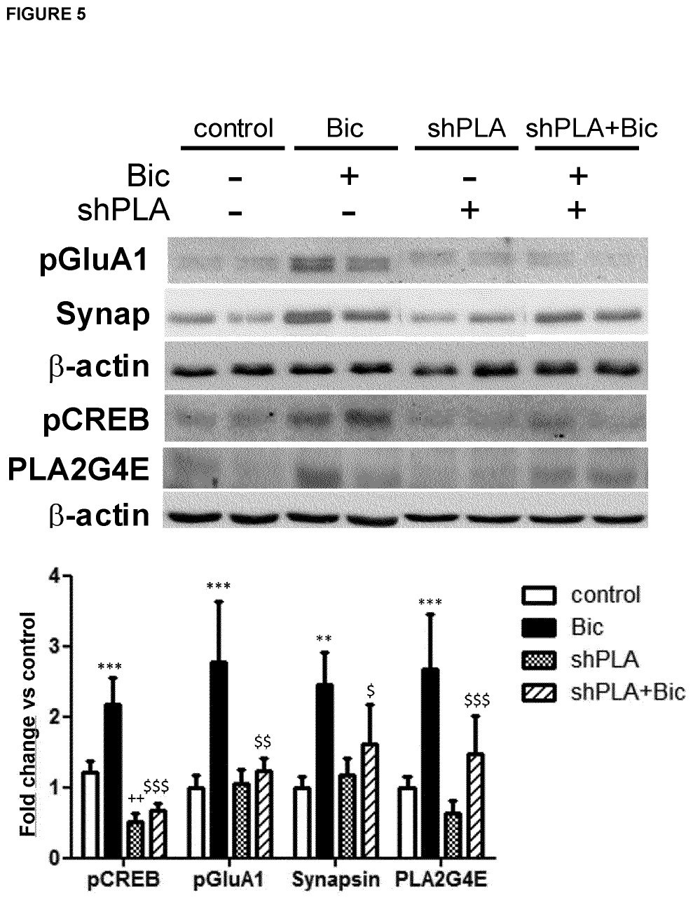

To demonstrate NMDA receptor activation, CREB activation (phosphorylation of CREB at the activator site residue Ser 133) was analyzed (Ginty et al., 1993 Science 260: 238-241). As depicted in FIG. 5 and described by several authors (Hardingham et al., 2002 Nat. Neurosci., 5: 405-414), we demonstrated that bicuculline (through the activation of NMDA receptors) caused a sustained CREB phosphorylation at Ser133 as well as an increase of AMPA receptor activation (Rao et al., 2006 Nat. Neurosci., 9: 887-895) which was analyzed measuring pGluA1 levels.

Synapsin I levels were also analyzed since this presynaptic protein increases in the hippocampus during long term potentiation (LTP) (Sato et al., 2000 Brain Res., 872: 219-222) and plays a fundamental role in the formation, maintenance and rearrangements of synaptic contacts (review in Cesca et al., 2010 Prog. Neurobiol., 91: 313-348). A significant increase of synapsin I was also observed in neuronal cultures activated with bicuculline.

PLA2G4E expression was next analyzed on the same conditions and, interestingly, we observed that it was strongly induced by bicuculline which indicates that neuronal activation indeed upregulated PLA2G4E expression.

The effect of chronic PLA2G4E knockdown using an AAV-shPLA2G4E was then analyzed. In neuronal primary cultures we demonstrated that the treatment with AAV-shPLA2G4E blocked bicuculline-induced PLA2G4E expression and effectively blocked the activation of CREB and GluA1 driven by bicuculline (FIG. 5). Likewise, acute PLA2G4E knockdown no longer increased synapsin I expression in response to bicuculline.

In summary, these data suggest that synapse formation and/or stability may be altered upon PLA2G4E knockdown.

Sequences of the disclosure
HUMAN Cytosolic phospholipase A2 epsilon (Isoform 1)
SEQ ID NO: 1
MSLQASEGCPGLGTNVFVPQSPQTDEEGSRSGRSFSEFEDTQDLDTPGLPPFCPMAPWGSEEGLSPCHLLTVRVI
RMKNVRQADMLSQTDCFVSLWLPTASQKKLRTRTISNCPNPEWNESFNFQIQSRVKNVLELSVCDEDTVTPDDHL
LTVLYDLTKLCFRKKTHVKFPLNPQGMEELEVEFLLEESPSPPETLVTNGVLVSRQVSCLEVHAQSRRRRKREKM
KDLLVMVNESFENTQRVRPCLEPCCPTSACFQTAACFHYPKYFQSQVHVEVPKSHWSCGLCCRSRKKGPISQPLD
CLSDGQVMTLPVGESYELHMKSTPCPETLDVRLGFSLCPAELEFLQKRKVVVAKALKQVLQLEEDLQEDEVPLIA
IMATGGGTRSMTSMYGHLLGLQKLNLLDCASYITGLSGATWTMATLYRDPDWSSKNLEPAIFEARRHVVKDKLPS
LFPDQLRKFQEELRQRSQEGYRVTFTDFWGLLIETCLGDERNECKLSDQRAALSCGQNPLPIYLTINVKDDVSNQ
DFREWEEFSPYEVGLQKYGAFIPSELFGSEFFMGRLVKRIPESRICYMLGLWSSIFSLNLLDAWNLSHTSEEFFH
RWTREKVQDIEDEPILPEIPKCDANILETTVVIPGSWLSNSFREILTHRSFVSEFHNFLSGLQLHTNYLQNGQFS
RWKDTVLDGFPNQLTESANHLCLLDTAFFVNSSYPPLLRPERKADLIIHLNYCAGSQTKPLKQTCEYCTVQNIPF
PKYELPDENENLKECYLMENPQEPDAPIVTFFPLINDTFRKYKAPGVERSPEELEQGQVDIYGPKTPYATKELTY
TEATFDKLVKLSEYNILNNKDTLLQALRLAVEKKKRLKGQCPS
Nucleotide Sequence encoding human cPLA2e isoform 1
SEQ ID NO: 2
ATGAGTCTCCAGGCCTCGGAAGGCTGTCCTGGCCTGGGAACTAATGTGTTTGTCCCACAGAGCCCACAAACGGAT
GAAGAAGGCAGCAGGTCAGGAAGAAGTTTCAGTGAGTTCGAGGATACACAGGACCTGGACACTCCTGGTCTCCCA
CCTTTCTGTCCTATGGCTCCTTGGGGCTCTGAGGAGGGGCTGTCTCCATGCCACCTGTTGACAGTGAGGGTCATC
CGGATGAAAAATGTCCGGCAGGCTGATATGCTGAGCCAGACAGACTGTTTTGTGAGCCTCTGGCTGCCCACCGCC
TCTCAGAAGAAGCTGAGGACAAGGACCATCTCCAACTGCCCAAATCCAGAGTGGAATGAAAGCTTCAACTTCCAG
ATCCAGAGCCGAGTGAAGAACGTGCTAGAGTTGAGTGTCTGTGATGAAGACACAGTGACACCAGATGACCATCTC
CTGACAGTTCTCTATGACCTCACCAAGCTCTGTTTCCGAAAGAAAACCCACGTGAAGTTTCCACTCAACCCGCAG
GGCATGGAAGAGCTGGAGGTGGAGTTCCTGCTGGAGGAGAGTCCCTCTCCACCTGAGACCCTCGTCACCAATGGC
GTGCTGGTGTCTCGACAAGTCTCCTGCCTGGAGGTTCATGCACAATCCAGGAGGCGGAGGAAGAGGGAGAAAATG
AAGGACCTCCTGGTGATGGTGAACGAATCCTTTGAGAACACCCAGCGTGTCCGGCCCTGCTTGGAACCCTGCTGC
CCAACCTCTGCCTGCTTCCAAACCGCTGCCTGCTTCCACTACCCCAAGTACTTCCAGTCCCAGGTGCACGTGGAA
GTGCCCAAGAGTCACTGGAGCTGTGGGCTTTGCTGCCGCTCTCGCAAGAAGGGCCCCATCAGCCAGCCCCTCGAC
TGCCTTTCCGATGGTCAGGTGATGACCCTGCCTGTGGGTGAGAGTTATGAATTACACATGAAGTCTACACCCTGC
CCTGAGACACTGGACGTGCGGCTGGGCTTCAGCCTGTGCCCAGCAGAGCTGGAGTTTCTGCAGAAGCGGAAGGTC
GTGGTGGCCAAGGCCCTGAAGCAGGTGCTGCAGCTGGAGGAAGACCTGCAGGAGGACGAGGTGCCGCTGATAGCC
ATCATGGCCACTGGGGGTGGAACAAGATCCATGACCTCCATGTATGGCCACCTGCTGGGGCTGCAGAAGCTGAAC
CTCCTGGACTGTGCCAGCTACATCACCGGTCTATCAGGGGCCACCTGGACCATGGCTACCTTGTACCGTGACCCT
GACTGGTCCTCCAAAAACTTGGAGCCTGCTATCTTTGAGGCTCGGAGACATGTGGTAAAGGACAAGCTACCCTCC
CTGTTCCCAGACCAGCTCCGCAAATTCCAGGAGGAGCTCCGGCAGCGCAGCCAGGAAGGCTACAGGGTCACCTTT
ACAGACTTCTGGGGCCTGCTGATAGAGACCTGCCTGGGGGACGAGAGAAATGAATGCAAACTGTCAGATCAGCGT
GCTGCTTTGAGCTGCGGCCAGAACCCCCTGCCCATCTACCTCACCATCAATGTCAAGGATGATGTAAGCAACCAG
GACTTCAGAGAGTGGTTCGAGTTCTCCCCCTACGAGGTGGGCCTGCAGAAGTATGGGGCCTTCATCCCCTCCGAG
CTCTTCGGCTCCGAGTTCTTCATGGGGCGGCTGGTGAAGAGGATCCCGGAGTCTCGAATCTGCTACATGCTAGGC
CTGTGGAGCAGCATCTTCTCCCTGAACCTGCTGGATGCCTGGAACCTGTCACACACCTCGGAGGAGTTTTTCCAC
AGGTGGACAAGGGAGAAAGTGCAGGACATCGAAGACGAGCCGATCCTGCCTGAAATCCCCAAATGTGATGCTAAC
ATCCTGGAGACCACGGTAGTGATCCCAGGGTCATGGCTGTCCAATTCTTTCCGAGAAATCCTTACCCATCGGTCC
TTCGTGTCTGAGTTTCACAACTTCCTGTCTGGGCTGCAGCTGCACACCAACTACCTCCAGAATGGCCAGTTCTCT
AGGTGGAAAGACACAGTGCTAGATGGTTTCCCAAACCAGCTGACCGAGTCCGCGAACCACCTGTGCCTGCTGGAC
ACTGCGTTCTTTGTCAACTCCAGCTACCCGCCCCTCCTCAGGCCAGAGCGAAAAGCCGACCTCATCATCCACCTC
AACTACTGTGCTGGGTCCCAGACAAAGCCCCTGAAACAAACCTGTGAGTACTGCACTGTGCAGAACATCCCCTTC
CCCAAATACGAGCTGCCAGATGAGAATGAAAATCTCAAGGAATGCTACCTGATGGAGAACCCCCAGGAACCCGAT
GCCCCCATCGTGACTTTCTTCCCACTCATCAATGACACTTTCCGAAAATACAAGGCACCAGGTGTAGAGCGAAGC
CCTGAGGAGCTGGAGCAGGGCCAGGTGGACATTTATGGTCCCAAAACTCCCTATGCCACCAAGGAGCTGACATAC
ACAGAGGCCACCTTTGACAAGCTGGTGAAACTCTCAGAGTATAACATCCTGAATAATAAGGACACTCTCCTCCAG
GCTCTGCGGCTCGCAGTGGAGAAGAAGAAGCGCCTGAAGGGCCAGTGTCCCTCCTAG
HUMAN Isoform 2 of Cytosolic phospholipase A2 epsilon
SEQ ID NO: 3
MATGGGTRSMTSMYGHLLGLQKLNLLDCASYITGLSGATWTMATLYRDPDWSSKNLEPAIFEARRHVVKDKLPSL
FPDQLRKFQEELRQRSQEGYRVTFTDFWGLLIETCLGDERNECKLSDQRAALSCGQNPLPIYLTINVKDDVSNQD
FREWFEFSPYEVGLQKYGAFIPSELFGSEFFMGRLVKRIPESRICYMLGLWSSIFSLNLLDAWNLSHTSEEFFHR
WTREKVQDIEDEPILPEIPKCDANILETTVVIPGSWLSNSFREILTHRSFVSEFHNFLSGLQLHTNYLQNGQFSR
WKDTVLDGFPNQLTESANHLCLLDTAFFVNSSYPPLLRPERKADLIIHLNYCAGSQTKPLKQTCEYCTVQNIPFP
KYELPDENENLKECYLMENPQEPDAPIVTFFPLINDTFRKYKAPGVERSPEELEQGQVDIYGPKTPYATKELTYT
EATFDKLVKLSEYNILNNKDTLLQALRLAVEKKKRLKGQCPS
Nucleotide Sequence encoding human cPLA2e isoform2
SEQ ID NO: 4
ATGGCCACTGGGGGTGGAACAAGATCCATGACCTCCATGTATGGCCACCTGCTGGGGCTGCAGAAGCTGAACCTC
CTGGACTGTGCCAGCTACATCACCGGTCTATCAGGGGCCACCTGGACCATGGCTACCTTGTACCGTGACCCTGAC
TGGTCCTCCAAAAACTTGGAGCCTGCTATCTTTGAGGCTCGGAGACATGTGGTAAAGGACAAGCTACCCTCCCTG
TTCCCAGACCAGCTCCGCAAATTCCAGGAGGAGCTCCGGCAGCGCAGCCAGGAAGGCTACAGGGTCACCTTTACA
GACTTCTGGGGCCTGCTGATAGAGACCTGCCTGGGGGACGAGAGAAATGAATGCAAACTGTCAGATCAGCGTGCT
GCTTTGAGCTGCGGCCAGAACCCCCTGCCCATCTACCTCACCATCAATGTCAAGGATGATGTAAGCAACCAGGAC
TTCAGAGAGTGGTTCGAGTTCTCCCCCTACGAGGTGGGCCTGCAGAAGTATGGGGCCTTCATCCCCTCCGAGCTC
TTCGGCTCCGAGTTCTTCATGGGGCGGCTGGTGAAGAGGATCCCGGAGTCTCGAATCTGCTACATGCTAGGCCTG
TGGAGCAGCATCTTCTCCCTGAACCTGCTGGATGCCTGGAACCTGTCACACACCTCGGAGGAGTTTTTCCACAGG
TGGACAAGGGAGAAAGTGCAGGACATCGAAGACGAGCCGATCCTGCCTGAAATCCCCAAATGTGATGCTAACATC
CTGGAGACCACGGTAGTGATCCCAGGGTCATGGCTGTCCAATTCTTTCCGAGAAATCCTTACCCATCGGTCCTTC
GTGTCTGAGTTTCACAACTTCCTGTCTGGGCTGCAGCTGCACACCAACTACCTCCAGAATGGCCAGTTCTCTAGG
TGGAAAGACACAGTGCTAGATGGTTTCCCAAACCAGCTGACCGAGTCCGCGAACCACCTGTGCCTGCTGGACACT
GCGTTCTTTGTCAACTCCAGCTACCCGCCCCTCCTCAGGCCAGAGCGAAAAGCCGACCTCATCATCCACCTCAAC
TACTGTGCTGGGTCCCAGACAAAGCCCCTGAAACAAACCTGTGAGTACTGCACTGTGCAGAACATCCCCTTCCCC
AAATACGAGCTGCCAGATGAGAATGAAAATCTCAAGGAATGCTACCTGATGGAGAACCCCCAGGAACCCGATGCC
CCCATCGTGACTTTCTTCCCACTCATCAATGACACTTTCCGAAAATACAAGGCACCAGGTGTAGAGCGAAGCCCT
GAGGAGCTGGAGCAGGGCCAGGTGGACATTTATGGTCCCAAAACTCCCTATGCCACCAAGGAGCTGACATACACA
GAGGCCACCTTTGACAAGCTGGTGAAACTCTCAGAGTATAACATCCTGAATAATAAGGACACTCTCCTCCAGGCT
CTGCGGCTCGCAGTGGAGAAGAAGAAGCGCCTGAAGGGCCAGTGTCCCTCCTAG
Nucleotide Sequence encoding murine PLA2G4E fused to a flag sequence
SEQ ID NO: 5
ATGCAGTCTATTCCACACTCCGATGAAGCAGACGTGGCTGGGATGACCCACGCCTCAGAAGGCCACCATGGCCTG
GGGACCAGCATGCTTGTCCCAAAGAACCCACAAGGGGAAGAAGACAGCAAGCTAGGAAGAAACTGCAGTGGATTT
GAAGATGCACAGGACCCACAGACTGCTGTGCCCTCCTCACCTTTACTTTCCATGGCTTCTTGCAGTTCTCAGGAG
GGGTCATCTCCATGCCATCTGTTGACAGTGAGGATCATTGGCATGAAAAACGTCCGGCAGGCTGATATACTGAGT
CAGACAGACTGCTTTGTGACCCTCTGGCTGCCTACTGCCTCTCAGAAGAAGCTGAAGACCAGAACCATCTCCAAC
TGCCTACACCCAGAGTGGGACGAAAGCTTCACCTTTCAGATCCAGACTCAAGTAAAGAATGTGCTAGAGCTGAGC
GTCTGTGACGAAGACACCCTGACACAAAATGACCATCTCTTGACAGTCCTCTATGACCTCTCTAAGCTTTGCCTC
CGGAATAAAACCCATGTGAAGTTCCCACTCAACCCAGAGGGCATGGAAGAACTGGAGGTGGAGTTCCTACTCGAA
GAGAATTTCTCCTCATCAGAGACCCTCATCACCAACGGCGTGCTGGTGTCTCGCCAAGTCTCTTGCCTGGAGGTT
CATGCAGAATCCAGGAGGCCGAGGAAGAGGAAGAAAAACAAAGACCTTCTGGTGATGGTGACAGACTCCTTCGAG
AACACCCAGCGTGTCCCGCCTTGCCAGGAGCCCTGCTACCCCAATTCTGCCTGCTTCCACTACCCCAAGTACTCC
CAGCCACAGCTTTACGCAGAGGCGCCTAAGAGCCACTGTAACTTTAGGCTTTGCTGCTGCGGAACACACAGGAAT
GACCCTGTCTGCCAGCCCCTCAATTGCCTTTCTGATGGCCAGGTGACAACCCTGCCTGTGGGAGAGAACTATGAG
CTACACATGAAGTCCTCACCCTGCTCTGACACACTGGATGTGCGGCTTGGATTCAGCCTGTGCCAGGAAGAGGTG
GAGTTTGTGCAGAAGCGGAAGATGGTGGTGGCCAAGACACTAAGTCAGATGCTGCAGCTGGAGGAAGGCCTGCAT
GAGGATGAGGTACCGATAATAGCCATCATGGCCACAGGAGGTGGCACAAGGTCTATGGTCTCCTTGTATGGCCAC
CTGCTGGGGTTGCAGAAGCTGAACTTTCTGGACGCTTCTACTTACATCACCGGCTTGTCAGGTGCAACCTGGACT
ATGGCTACCTTGTACAGTGATCCTGAGTGGTCCTCCAAAAACCTGGAGACTGTTGTCTTTGAGGCCCGGAGACAT
GTTGTCAAAGACAAGATGCCTGCCCTGTTCCCAGATCAGCTCTACAAATGGCGAGAGGACCTCCAAAAGCATAGC
CAGGAGGGCTATAAGACCACGTTTACAGACTTTTGGGGCAAGCTGATCGAGTACAGTCTGGGAGATAAAAAAAAC
GAATGCAAGCTGTCAGATCAGCGAGCTGCTCTGTGCAGGGGACAGAACCCTCTGCCCATCTACCTCACCATCAAT
GTCAAGGATGATGTAAGCAACCAGGATTTCAGAGAATGGTTCGAGTTCTCCCCCTACGAGGTGGGCATGCAGAAG
TACGGAGCCTTCATCCCCAGCGAGTTATTTGGCTCCGAGTTCTTCATGGGGCGGCTGATGAAGAGGATTCCTGAG
CCGGAGATGTGCTACATGCTAGGGTTGTGGAGTAGCATCTTTTCCCTGAACCTGCTTGATGCCTGGAATTTGTCT
CACACCTCAGAGGAGTTTTTCTATAGGTGGACAAGGGAGAGACTGCATGACATCGAAGATGATCCCATCCTGCCT
GAAATCCCTAGGTGTGACGATAACCCCCTAGAGACCACAGTAGTGATCCCAACGACATGGCTGTCCAACACCTTC
CGAGAAATCCTCACACGCAGGCCCTTCGTGTCTGAGTTCCACAACTTCCTGTACGGGATGCAGCTGCATACTGAC
TACTTACAGAACAGGCAGTTCTCTATGTGGAAAGACACAGTACTGGACACCTTCCCAAACCAGCTGACACAGTTT
GCAAAACACCTGAACCTGCTGGACACTGCGTTCTTTGTCAACTCCAGCTACGCACCCCTCCTTAGGCCAGAGAGA
AAAGTCGACCTTATCATCCACCTCAATTACTGCGCAGGATCCCAGACAAAGCCCCTGAAACAAACCTGTGAGTAC
TGTACCGAGCAGAAGATCCCCTTCCCCAGCTTCTCCATCCTGGAAGATGACAACAGTCTCAAGGAGTGCTACGTG
ATGGAGAATCCCCAGGAGCCCGACGCCCCCATCGTGGCTTACTTCCCACTCATCAGTGACACCTTCCAGAAGTAC
AAGGCTCCAGGTGTAGAGCGAAGTCCTGACGAGCTGGAACTGGGCCAGCTGAACATCTATGGACCAAAGTCTCCC
TATGCCACCAAGGAGCTGACGTACACAGAGGCCGCCTTCGACAAGCTGGTGAAGCTCTCAGAATATAATATCCTC
AATAACAGAGATAAGCTCATTCAGGCCTTGAGACTAGCAATGGAGAAGAAACGCATGAGGAGCCAGTGTCCCTCC
GCGGCCGCAGGAGGTGGAGGTGACTACAAGGATGACGATGACAAGTGA
Forward primer fwPLA2G4E
SEQ ID NO: 6
ATGGTGACAGACTCCTTCGAG
Reverse primer rvPLA2G4E
SEQ ID NO: 7
CCTCTGCGTAAAGCTGTGG
shRNA for PLA2G4E(shPLA2G4E)
SEQ ID NO: 8
GGTCTATGGTCTCCTTGTATCAAGAGTACAAGGAGACCATAGACC

1. A nucleic acid construct that comprises a nucleotide sequence encoding a cytosolic phospholipase A2 epsilon (cPLA2e).

2. The nucleic acid construct of claim 1, wherein the cPLA2e is a human cPLA2e; typically human cPLA2e of SEQ ID NO: 1 or SEQ ID NO:3, or a variant human cPLA2e having at least 70% sequence identity with respect to human cPLA2e SEQ ID NO:1 or SEQ ID NO:3.

3. The nucleic acid construct of claim 2, wherein nucleotide sequence encoding cPLA2e is SEQ ID NO:2 or SEQ ID NO:4.

4. The nucleic acid construct of any of claims 1-3, wherein said nucleic acid construct further comprises a promoter operably-linked to the nucleotide sequence encoding a cPLA2e.

5. The nucleic acid construct of claim 4, wherein said promoter is a neuronal-specific promoter; preferably said promoter is a SYN1 promoter or hybrid SYN1 promoter.

6. The nucleic acid construct of any of claims 1-5, wherein said nucleic acid construct further comprises a polyadenylation signal sequence; preferably a polyadenylation signal sequence of bovine growth hormone gene.

7. The nucleic acid construct of any of claims 1-6, wherein said nucleic acid construct further comprises a 5′ITR and a 3′ITR sequences; preferably a 5′ITR and a 3′ITR sequences of an adeno-associated virus, more preferably a 5′ITR and a 3′ITR sequences from the AAV2 serotype.

8. A vector that comprises a nucleic acid construct of any of claims 1-7.

9. The vector of claim 8, wherein the vector is a viral vector.

10. The vector of claim 9, wherein the vector is an AAV vector.

11. The vector of claim 10, wherein the vector comprises a nucleic acid construct of claim 2.

12. A viral particle that includes a nucleic acid construct of any of claims 1-7 or a vector of claims 8-11.

13. The viral particle of claim 12, wherein said viral particle is selected among AAV particles; preferably including capsid proteins selected from the group consisting of AAV2, AAV5, AAV9, and AAV TT serotypes.

14. A host cell comprising a nucleic acid construct of any of claims 1-7, or a vector of claims 8-11.

15. A process for producing viral particles comprising:
a) culturing a packaging cell comprising a nucleic acid construct of any of claims 1-7 or a vector of claims 8-11 in a culture medium; and
b) harvesting the viral particles from the cell culture supernatant and/or inside the cells.

16. A pharmaceutical composition comprising a nucleic acid construct of any of claims 1-7, a vector of any of claims 8-11, a viral particle of any of claims 12-13, or a host cell of claim 14; and a pharmaceutically acceptable carrier or excipient.

17. A pharmaceutical composition comprising a nucleic acid construct of any of claims 1-7, a vector of any of claims 8-11, a viral particle of any of claims 12-13, a host cell of claim 14, or pharmaceutical composition of claim 16 for use as a medicament.

18. A cPLA2e inducing agent for use as a medicament.

19. A cPLA2e inducing agent for use in the treatment of cognitive disorders and/or diseases associated with cognitive disorders in a subject in need thereof.

20. The cPLA2e inducing agent for use of claim 20, wherein the disease is dementia.

21. The cPLA2e inducing agent for use of claim 20, wherein the disease is an age-related dementia or Alzheimer's disease.

22. The cPLA2e inducing agent for use of any of claims—19-21, wherein said cPLA2e inducing agent is a nucleic acid construct of any of claims 1-7, a vector of any of claims 8-11, a viral particle of any of claims 12-13, a host cell of claim 14, or pharmaceutical composition of claim 16.

23. The cPLA2e inducing agent for use of any of claims 19-21, wherein said cPLA2e inducing agent is a protein with cPLA2e activity.

24. The cPLA2e inducing agent for use of claim 23, wherein said protein with cPLA2e activity is a protein comprising or consisting of SEQ ID NO:1 or SEQ ID NO:3 or a variant with at least 70% sequence identity thereto.

25. A method for identifying a compound as a candidate for the treatment a cognitive disorder and/or disease associated with a cognitive disorder which comprises the steps of:
a. contacting the compound with mammalian assay cells;
b. checking whether an effect related to cPLA2e induction or increase is produced;
c. identifying the compound as a candidate for the treatment of a cognitive disorder and/or disease associated with a cognitive disorder if such effect is produced.
